(19) United States
(12) Patent Application Publication
(10) Pub. No.: US 2019/0059433 A1
(43) Pub. Date: Feb. 28, 2019

(54) COMPOSITIONS AND METHODS FOR RESTORING METABOLIC HEALTH

(71) Applicant: Wisconsin Alumni Research Foundation, Madison, WI (US)

(72) Inventors: Dudley William Lamming, JR., Madison, WI (US); Nicole E. Cummings, Madison, WI (US); Lisa Davis, Middleton, WI (US)

(21) Appl. No.: 16/057,935

(22) Filed: Aug. 8, 2018

Related U.S. Application Data
(60) Provisional application No. 62/549,639, filed on Aug. 24, 2017.

(51) Int. Cl. A23L 33/175; A61P 3/00; A61P 3/08; A61K 31/423; A61K 31/351; A23L 33/20; A23K 20/147; A23K 50/50

(52) U.S. Cl. CPC A23L 33/175; A61P 3/00; A61P 3/08; A61K 31/423; A23V 2002/00; A23L 33/20; A23K 20/147; A23K 50/50; A61K 31/351

(57) ABSTRACT

A method of improving metabolic health in a mammalian subject in need of improvement in metabolic health includes reducing the mammalian subject's consumption of branched chain amino acids (BCAAs) by the mammalian subject consuming a reduced BCAA daily diet. Also included is a method of reducing a mammalian subject's consumption of BCAAs by the mammalian subject consuming a reduced BCAA meal replacement at least once per day. Also included is a method of feeding a mammalian subject in need of weight reduction by the mammalian subject consuming a low protein daily diet supplemented with leucine. Also included is a method of improving metabolic health in a mammalian subject in need of improvement in metabolic health by reducing the mammalian subject's consumption either histidine alone or of both phenylalanine and tyrosine by the mammalian subject consuming a reduced amino acid daily diet.

### CROSS-REFERENCE TO RELATED APPLICATIONS

This application claims priority to U.S. Provisional Application 62/549,639 filed on Aug. 24, 2017, which is incorporated herein by reference in its entirety.

### STATEMENT REGARDING FEDERALLY SPONSORED RESEARCH & DEVELOPMENT

This invention was made with government support under AG041765, AG050135 and CA014520 awarded by the National Institutes of Health. The government has certain rights in the invention.

### FIELD OF THE DISCLOSURE

The present disclosure is related to methods of improving metabolic health in mammalian subjects, particularly mammalian subjects that are obese, overweight, or would otherwise benefit from weight loss.

### BACKGROUND

Over the last four decades, the body mass index (BMI) of the population of the United States and much of the developed world has increased dramatically. In the United States, more than 2 in 3 adults are now considered to be overweight or obese; a similar proportion of males in the European Union are likewise afflicted. Obesity is associated with an increased risk of many diseases, most notably type 2 diabetes, which now affects over 29 million Americans (12.3% of adults over the age of 20). An additional 86 million Americans over the age of 20 are estimated to have pre-diabetes, making this disease one of the most urgent health care problems facing the United States. Obesity and type 2 diabetes are both risk factors for cardiovascular disease, one of the top causes of mortality in the United States, and Alzheimer's disease, a disease for which there is no effective preventive or cure.

Dietary interventions to control obesity and to control or prevent type 2 diabetes could be highly effective and affordable, but the most obvious form, reduced calorie diets, are notoriously difficult to sustain. Altering the macronutrient composition of the diet while keeping the total number of calories constant is an intriguing alternative that may be more sustainable. High protein, low carbohydrate diets such as the Atkins diet have received significant attention due to the promise of rapid weight loss while not restricting calories. However, studies have found that high protein consumption is correlated with insulin resistance, diabetes, and mortality in both mice and humans; moreover, high protein diets may not be sustainable in the long term. Conversely, low protein diets are associated with increased survival in both rodents and humans. A recent randomized controlled trial found that a low protein diet promotes leanness and decreases fasting blood glucose in humans.

It has been demonstrated that a low protein diet promotes metabolic health in young rodents, reducing the accumulation of white adipose tissue and increasing glucose tolerance and insulin sensitivity. What is needed are alternative methods for improving metabolic health in mammals.

### BRIEF SUMMARY

In one aspect, a method of improving metabolic health in a mammalian subject in need of improvement in metabolic health comprises reducing the mammalian subject's consumption of branched chain amino acids (BCAAs) by the mammalian subject consuming a reduced BCAA daily diet, wherein the reduced BCAA daily diet has the following characteristics:
the percentage of calories from BCAAs consumed during the course of the day are reduced compared to the percentage of calories from BCAAs in a standard daily diet for the mammalian subject,
the daily intake of BCAAs in the reduced BCAA daily diet provides at least the recommended daily allowance of isoleucine, leucine and valine for the mammalian subject
the reduced BCAA daily diet includes all essential amino acids, and optionally non-essential amino acids,
the percentage of calories from non-BCAAs in the reduced BCAA daily diet are not substantially reduced compared to the percentage of calories from non-BCAAs in the standard daily diet for the mammalian subject, and
the reduced BCAA daily diet has at least the same number of calories as the standard daily diet for the mammalian subject, and

wherein the individual is not suffering from maple syrup urine disease.

In another aspect, a method of improving metabolic health in a mammalian subject in need of improvement in metabolic health due to excess weight or poor control of blood glucose levels comprises administering to the mammalian subject a compound that inhibits at least a portion of the gastrointestinal absorption of one or more branched chain amino acids.

In an aspect, a method of feeding a mammalian subject in need of weight reduction comprises reducing the mammalian subject's consumption of BCAAs by the mammalian subject consuming a reduced BCAA meal replacement at least once per day,
wherein the percentage of calories from BCAAs in the reduced BCAA meal replacement are reduced compared to the percentage of calories from BCAAs in a standard meal for the mammalian subject, and
wherein the reduced BCAA meal replacement includes the essential amino acids histidine, lysine, methionine, phenylalanine, threonine, and tryptophan.

In another aspect, a method of feeding a mammalian subject in need of weight reduction comprises

the mammalian subject consuming a low protein daily diet supplemented with leucine, wherein the total amount of leucine in the low protein diet supplemented with leucine is greater than or equal to 1.15 wt % of the total calories and/or comprises a daily consumption of 6 g or more of leucine per day,

wherein the low protein diet comprises less than 9% of total calories from protein, and wherein the normal diet comprises greater than 15% of total calories from protein.

In a further aspect, a method of improving metabolic health in a mammalian subject in need of improvement in metabolic health, comprises

reducing the mammalian subject's consumption of either histidine alone or of both phenylalanine and tyrosine by the mammalian subject consuming a reduced amino acid daily diet, wherein the reduced amino acid daily diet has the following characteristics:
a percentage of calories from either histidine alone or of both phenylalanine and tyrosine consumed during the course of the day are reduced compared to a percentage of calories from either histidine alone or of both phenylalanine and tyrosine in a standard daily diet for the mammalian subject,
the daily intake of either histidine alone or of both phenylalanine and tyrosine in the reduced amino acid daily diet provides at least the recommended daily allowance of histidine, phenylalanine and tyrosine for the mammalian subject
the reduced amino acid daily diet includes all essential amino acids, and optionally non-essential amino acids,
the percentage of calories from amino acids other than histidine or phenylalanine and tyrosine in the reduced BCAA daily diet are not substantially reduced compared to their percentages in the standard daily diet for the mammalian subject, and
   the reduced amino acid daily diet has at least the same number of calories as the standard daily diet for the mammalian subject.

In yet another aspect, a method of feeding a mammalian subject in need of weight reduction comprises

reducing the mammalian subject's consumption of either histidine alone or of both phenylalanine and tyrosine by the mammalian subject consuming a reduced amino acid meal replacement at least once per day, wherein the percentage of calories from histidine or of both phenylalanine and tyrosine in the reduced amino acid meal replacement are reduced compared to the percentage of calories from histidine alone or of both phenylalanine and tyrosine in a standard meal for the mammalian subject, and wherein the reduced amino acid meal replacement includes all essential amino acids.

### BRIEF DESCRIPTION OF THE DRAWINGS

FIG. 1 is a schematic representation of the experimental plan for Example 1; mice were preconditioned with a Western diet for 12 weeks and then randomized to the five experimental groups shown, while chow fed Control mice were placed on an amino acid defined Control diet.

FIG. 2 shows weight in each experimental group (n=12 mice/group) from Example 1 as a function of time.

FIG. 3 shows food intake assessed over a 4 day period in home cages for 2 and 10 weeks following the start of the specified dietary intervention. (n=6-7 cages/group, means with the same letter are not significantly different from each other (Tukey-Kramer test following ANOVA, p<0.05)).

FIG. 4 shows adipose and lean mass of mice in each experimental group (n=12 mice/group) as function of time after dietary switch.

FIG. 5 shows body composition plotted as fat and lean fraction determined 9 weeks after the start of the specified diet intervention (n=11-12 mice/group, means with the same letter are not significantly different from each other (Tukey-Kramer test following ANOVA, p<0.05)).

FIG. 6 shows paraffin-embedded skin samples collected at necropsy approximately 15 weeks following the start of the specified dietary intervention. The samples were sectioned and H&E stained.

FIG. 7 shows the thickness of dermal white adipose tissue (dWAT) non-anagen stage skin samples, measuring from muscle to dermis; scale bar=200 μM (n=5-7 mice/group, *=p<0.05, Dunnett's test following ANOVA, **=p<0.05, Bonferroni's test).

FIG. 8 shows the mass of epididymal white adipose tissue (eWAT) collected at necropsy and weighed (n=11-12 mice/group, *=p<0.05, Dunnett's test following ANOVA, **=p<0.05, Bonferroni's test).

FIG. 9 shows OCT-embedded liver samples which were cryosectioned and stained with Oil-Red-O.

FIG. 10 shows quantification of droplet size. scale bar=200 μM (n=3 mice/group, *=p<0.05 vs. WD Control AA, Dunnett's test following ANOVA, p<0.05).

FIG. 11 shows lipogenic gene expression measured in the livers of mice on the specified diets fasted overnight (n=5-6 mice/group, *=p<0.05 vs. WD Control AA, Dunnett's test following 2-way repeated measures ANOVA).

FIG. 12 shows glucose tolerance of mice in each diet group after 3 weeks of dietary intervention (n=12 mice/group, means with the same letter are not significantly different from each other (Tukey-Kramer test following ANOVA, p<0.05)).

FIG. 13 shows a glucose tolerance test conducted at 9 weeks after the start of the dietary interventions (n=10-12/group; for AUC, means with the same letter are not significantly different from each other (Tukey-Kramer test following ANOVA, p<0.05).

FIG. 14 shows an insulin tolerance test conducted at 4 weeks after the start of the dietary interventions (n=10-12/group; for AUC, means with the same letter are not significantly different from each other (Tukey-Kramer test following ANOVA, p<0.05).

FIG. 15 shows an insulin tolerance test conducted at 10 weeks after the start of the dietary interventions (n=10-12/group; for AUC, means with the same letter are not significantly different from each other (Tukey-Kramer test following ANOVA, p<0.05).

FIGS. 16 A-C show data for mice fasted overnight. In FIG. 16 A) blood glucose and in FIG. 16B) insulin were measured, and in FIG. 16 C) the HOMA2-IR was calculated after 5 weeks on the specified diets (n=5-7 mice/group; Tukey-Kramer test following ANOVA, p<0.05).

FIG. 17 shows the respiratory exchange ratio (RER) using metabolic chambers approximately 7-8 weeks after the start of the dietary intervention ((n=5-8 mice/group, means with the same letter are not significantly different from each other (Tukey-Kramer test following ANOVA, p<0.05)).

FIG. 18 shows the spontaneous activity measured using metabolic chambers approximately 7-8 weeks after the start of the dietary intervention ((n=5-8 mice/group, means with the same letter are not significantly different from each other (Tukey-Kramer test following ANOVA, p<0.05)).

FIG. 19 shows the energy expenditure measured using metabolic chambers approximately 7-8 weeks after the start of the dietary intervention ((n=5-8 mice/group, means with the same letter are not significantly different from each other (Tukey-Kramer test following ANOVA, p<0.05)).

FIG. 20 is a schematic representation of the experimental plan for Example 2; mice were preconditioned with a Western diet (WD) for 12 weeks and then randomized to the four experimental groups shown, while chow fed Control mice were placed on an amino acid defined Control diet.

FIG. 21 shows weight of mice in each experimental group (n=12 mice/group) as a function of time since diet switch.

FIG. 22 shows adipose and lean mass of mice in each experimental group (n=12 mice/group) as a function of time since diet switch.

FIG. 23 shows paraffin-embedded skin samples collected after feeding mice the indicated diets for approximately 14 weeks. The skin samples were sectioned, H&E stained and the thickness of dermal white adipose tissue (dWAT) was quantified for non-anagen stage skin samples, measuring from muscle to dermis; scale bar=200 μM (n=6 mice/group, *=p<0.05 vs. WD Control AA, Dunnett's test following ANOVA, p<0.05)).

FIG. 24 shows OCT-embedded liver samples which were cryosectioned and stained with Oil-Red-O, and droplet size was quantified; scale bar=200 μM (n=3 mice/group, *=p<0.05 vs. WD Control AA, Dunnett's test following ANOVA, p<0.05)).

FIG. 25 shows a comparison of body composition at diet intervention start and 12 weeks later.

FIG. 26 shows food intake measured two weeks after special diet feeding start (n=12 mice/group, means with the same letter are not significantly different from each other (Tukey-Kramer test following ANOVA, p<0.05)).

FIG. 27 shows a glucose tolerance test conducted 3 weeks after the start of the dietary interventions (n=12-16/group; for AUC, means with the same letter are not significantly different from each other (Tukey-Kramer test following ANOVA, p<0.05).

FIG. 28 shows an insulin tolerance test conducted 4 weeks after the start of the dietary interventions (n=12-16/group; for AUC, means with the same letter are not significantly different from each other (Tukey-Kramer test following ANOVA, p<0.05).

FIG. 29 shows a glucose tolerance test conducted 9 weeks after the start of the dietary interventions (n=12-16/group; for AUC, means with the same letter are not significantly different from each other (Tukey-Kramer test following ANOVA, p<0.05).

FIG. 30 shows an insulin tolerance test conducted 10 weeks after the start of the dietary interventions (n=12-16/group; for AUC, means with the same letter are not significantly different from each other (Tukey-Kramer test following ANOVA, p<0.05).

FIG. 31 shows blood glucose measured for mice fasted overnight.

FIG. 32 shows insulin levels measured for mice fasted overnight.

FIG. 33 shows the HOMA2-IR calculated after 5 weeks on the specified diets (n=3-7 mice/group; Dunnett's test following ANOVA, *=p<0.05, #=p<0.12).

FIG. 34 shows the results of an ex vivo insulin secretion assay to assess insulin secretion per islet in response to low (1.7 mM) and high (16.7 mM) glucose in mice kept on the indicated diets for approximately 14 weeks (n=6 mice/group, *=p<0.05 vs WD Control AA, Dunnett's test following ANOVA).

FIG. 35 shows an ex vivo insulin secretion assay to assess islet insulin content in response to low (1.7 mM) and high (16.7 mM) glucose in mice kept on the indicated diets for approximately 14 weeks (n=6 mice/group, *=p<0.05 vs WD Control AA, Dunnett's test following ANOVA).

FIG. 36 shows the mitochondrial membrane potential measured in ex vivo isolated pancreatic islets stimulated with low (2 mM) and high (20 mM) glucose levels (n=44-74 islets per group, Dunnett's test following ANOVA, *=p<0.05).

FIG. 37 shows food intake over a 24-hour period measured after mice had been on the diets for approximately 12 weeks using metabolic chambers (n=2-5 mice/group, means with the same letter are not significantly different from each other (Bonferroni test, p<0.05).

FIG. 38 shows food intake over a 24-hour period measured after mice had been on the diets for approximately 6 weeks in metabolic chambers at the specified time (n=3-4 mice/group, means with the same letter are not significantly different from each other, Bonferroni test, p<0.05).

FIG. 39 shows respiratory exchange ratio (RER) measured using metabolic chambers approximately 12 weeks after the start of the dietary intervention (n=4-5 mice/group, means with the same letter are not significantly different from each other (Tukey-Kramer test following ANOVA, p<0.05)).

FIG. 40 shows respiratory exchange ratio (RER) measured using metabolic chambers approximately 6 weeks after the start of the dietary intervention ((n=5-8 mice/group, means with the same letter are not significantly different from each other (Tukey-Kramer test following ANOVA, p<0.05)).

FIG. 41 shows spontaneous activity measured using metabolic chambers approximately 12 weeks after the start of the dietary intervention (n=4-5 mice/group, means with the same letter are not significantly different from each other (Tukey-Kramer test following ANOVA, p<0.05)).

FIG. 42 shows spontaneous activity measured using metabolic chambers approximately 6 weeks after the start of the dietary intervention ((n=5-8 mice/group, means with the same letter are not significantly different from each other (Tukey-Kramer test following ANOVA, p<0.05)).

FIG. 43 shows energy expenditure measured using metabolic chambers approximately 12 weeks after the start of the dietary intervention (n=4-5 mice/group, means with the same letter are not significantly different from each other (Tukey-Kramer test following ANOVA, p<0.05)).

FIG. 44 shows energy expenditure measured using metabolic chambers approximately 6 weeks after the start of the dietary intervention ((n=5-8 mice/group, means with the same letter are not significantly different from each other (Tukey-Kramer test following ANOVA, p<0.05)).

FIG. 45 shows FGF21 measured in the plasma of mice sacrificed following an overnight fast (n=4 mice/group, means with the same letter are not significantly different from each other (Tukey-Kramer test following ANOVA, p<0.05)).

FIG. 46 shows the weight of DIO mice switched to the indicated diet at time 0 (n=8/group).

FIG. 47 shows the change in fat and lean mass in mice placed on each diet for 7 days (n=8 mice/group, *=p<0.05 vs. WD Control AA, Dunnett's test following ANOVA, p<0.05).

FIG. 48 shows respiratory exchange ratio (RER), spontaneous activity, and energy expenditure measured 1 week after the diet switch (n=6-8 mice/group, Dunnett's test following ANOVA, *=p<0.05).

FIG. 49 shows energy expenditure over the course of a 24 hour cycle starting at approximately 10 am (n=8 mice/group; dark outline of symbol indicates p<0.05 vs. WD Control AA (Dunnett's test following two-way repeated measures ANOVA).

FIG. 50 shows FGF21 measured in the plasma of mice sacrificed following an overnight fast (n=6 mice/group, means with the same letter are not significantly different from each other (Tukey-Kramer test following ANOVA, p<0.05)).

FIG. 51 shows paraffin-embedded brown adipose tissue collected, sectioned, and H&E stained. Lipid droplet size was quantified; scale bar=200 μM (n=6 mice/group, Tukey-Kramer test following ANOVA, *=p<0.05).

FIG. 52 shows Bmp8 gene expression was assessed by qPCR in the brown adipose tissue of mice on the specified diets fasted overnight (n=6 mice/group, *=p<0.05 vs. WD Control AA, Dunnett's test following ANOVA).

FIG. 53 shows specifically reducing either dietary Isoleucine or dietary Valine improves glucose tolerance. GTT was performed 3 weeks after diet switch; n=9 mice/grp; means with the same letter are not significantly different (Tukey-Kramer test following ANOVA, p<0.05).

FIG. 54 shows specifically reducing dietary isoleucine improves hepatic insulin sensitivity. Hyperinsulinemic-euglycemic clamp of mice fed indicated diets for five weeks (n=6 Control, 7 Low Ile, *=p<0.05, t-test.

FIG. 55 shows specifically reducing dietary Isoleucine reduces fat mass gain.

FIG. 56 shows adding back normal levels of all three BCAAs to a low amino acid diet blocks the effect of a Low AA diet on fat mass gain. Specifically adding back (supplementing) a Low AA diet with Leucine, but not Isoleucine or Valine, potently inhibits fat mass gain.

FIG. 57 shows reduction of dietary Isoleucine is necessary for the effect of a LP diet on glucose tolerance. GTT was performed 3 weeks after diet switch; n=10-12 mice/grp; means with the same letter are not significantly different (Sidak test following ANOVA, p<0.05 vs. Low AA fed mice).

FIG. 58 shows specifically reducing either dietary isoleucine or valine recapitulates the effects of reducing all three BCAAs on the weight of diet-induced obese mice.

FIG. 59 shows specifically reducing either isoleucine or valine reduces fat mass, and isoleucine restriction also reduces lean mass, in diet-induced obese mice.

FIG. 60 shows adding back leucine to a low amino acid diet results in rapid weight loss; adding back isoleucine slows weight loss.

FIG. 61 shows specifically restricting histidine (His) or both phenylalanaine and tyrosine (Phe/Tyr) by 67% results in weight loss in obese mice consuming a high-fat, high sucrose Western diet.

In all cases, error bars represent standard error.

The above-described and other features will be appreciated and understood by those skilled in the art from the following detailed description, drawings, and appended claims.

### DETAILED DESCRIPTION

The inventors have demonstrated that reducing dietary branched chain amino acids (BCAAs) is sufficient to promote metabolic health in obese, metabolically disadvantaged mammals. Reducing dietary BCAA consumption can be used, for example, in the treatment of insulin-resistant obesity in humans and domestic companion animals, including cats and dogs. Importantly, the method does not rely upon restriction of calories which is the basis of most existing weight loss plans and diet systems.

Specifically, the inventors have demonstrated that reducing dietary levels of the BCAAs can restore metabolic health to diet-induced obese mice preconditioned with a Western diet. Specifically reducing dietary BCAAs rapidly reverses diet-induced obesity, inducing weight loss primarily through a reduction in adipose mass, and improving glucoregulatory control, even if mice continue to consume a high-calorie, high-fat, high-sugar diet. Reducing dietary BCAAs induces a transient increase in FGF21 but a sustained increase in energy expenditure.

As used herein, BCAAs are isoleucine, leucine and valine. Non-BCAAs include all essential and non-essential amino acids other than isoleucine, leucine and valine. There are 9 standard essential amino acids: isoleucine, leucine, valine, histidine, lysine, methionine, phenylalanine, threonine, and tryptophan. The non-essential standard amino acids include alanine, arginine, asparagine, aspartic acid, cysteine, glutamic acid, glutamine, glycine, proline, serine, and tyrosine. The compositions and methods herein can also include other naturally occurring amino acids.

In an aspect, a method of improving metabolic health in a mammalian subject in need of improvement in metabolic health comprises reducing the mammalian subject's consumption of BCAAs by the mammalian subject consuming a reduced BCAA daily diet. Exemplary mammalian subjects in need of improved metabolic health include obese and overweight subjects, subjects with elevated blood glucose levels such as fasting blood glucose levels, subjects with pre-diabetes, subjects with diabetes, subjects with impaired glucose tolerance, subjects with insulin resistance, subjects suffering from fatty liver disease, subjects suffering from cardiovascular disease, or a combination thereof. Additional subjects include middle aged subjects as midlife obesity is a risk factor for diseases such as Alzheimer's disease and cardiovascular disease.

As used herein a standard daily diet for a mammalian subject is the normal daily diet consumed by the mammalian subject prior to consuming the reduced BCAA daily diet, or a reference daily diet based on the average caloric intake and average protein consumption for the mammalian subject, adjusted for weight, and optionally age.

As used herein, a reduced BCAA daily diet has one or more, specifically all, of the following characteristics:
a percentage of calories from BCAAs consumed during the course of the day are reduced compared to a percentage of calories from BCAAs in a standard daily diet for the mammalian subject,
the daily intake of BCAAs in the reduced BCAA daily diet provides at least the recommended daily allowance of isoleucine, leucine and valine for the mammalian subject,
the reduced BCAA daily diet includes all essential amino acids, and optionally non-essential amino acids,
the percentage of calories from non-BCAAs in the reduced BCAA daily diet are not substantially reduced compared to the percentage of calories from non-BCAAs in the standard daily diet for the mammalian subject, and/or the reduced BCAA daily diet has at least the same number of calories as the standard daily diet for the mammalian subject.

In an aspect, the reduced BCAA daily diet is reduced in leucine, isoleucine and valine; the reduced BCAA daily diet is reduced in both isoleucine and valine only; or the reduced BCAA daily diet is reduced in isoleucine only; or the reduced BCAA daily diet is reduced in valine only.

As used herein, improving metabolic health is defined as improving one or more factors associated with metabolic health such as blood pressure, triglycerides, LDL-cholesterol, HDL-cholesterol, total cholesterol, fasting plasma glucose, fasting plasma insulin, insulin sensitivity, homeostasis model of assessment, body mass index. Metabolically unhealthy individuals can be normal weight or overweight individuals, and may not have been diagnosed with a particular disease, or may have been diagnosed by a physician with pre-diabetes, diabetes, and/or metabolic syndrome. These indicators of metabolic health may or may not be linked to an increased risk of conditions including heart disease, stroke, diabetes, pre-diabetes, dementia, and/or Alzheimer's disease.

As used herein, a daily diet is the diet consumed by a subject over the course of a 24 hour period, and for a human generally consists of three meals (e.g., breakfast, lunch and dinner) and optional snacks. Other mammals may consume more or fewer meals over the course of a day. The BCAA and non-BCAA content of each individual meal may vary, however, in the methods described herein, the total dietary BCAAs consumed over a 24 hour period is reduced. Similarly, each individual meal may not include all essential amino acids, however, over the course of a 24 hour period, the diet includes all essential amino acids.

Also as used herein, a standard daily diet is the diet of the mammalian subject prior to consuming the reduced BCAA diet described herein, such as the diet consumed the day or week, days or weeks before the reduced BCAA diet is started. The standard daily diet may be a reference daily diet based on the average caloric intake and average protein consumption for the mammalian subject, adjusted for weight, and optionally age. The standard diet may be a healthy diet, wherein the subject is simply consuming more calories than needed for their activity level, resulting in an imbalance in metabolic health. Alternatively, the standard diet may be an unhealthy diet wherein the subject is not eating the proper balance of foods to maintain good metabolic health.

The percentage of calories from BCAAs in the reduced BCAA daily diet are reduced compared to the percentage of calories from BCAAs in a standard daily diet for the mammalian subject. Exemplary reductions include at least 25%, 40%, 50%, 67% or more compared to the percentage of calories from BCAAs in a standard daily diet for the mammalian subject.

In an aspect, the method further comprises determining the BCAA content in the standard daily diet for the mammalian subject (e.g., by determining BCAA consumption over 1 or more days prior to starting the reduced BCAA daily diet) and reducing the BCAA content in the standard daily diet to provide the reduced BCAA diet. In the reduced BCAA diet, one or more meals and/or snacks may include reduced BCAAs, so long as the BCAAs consumed over a 24 hour period are reduced compared to the levels in the standard diet for the mammalian subject.

In another aspect, when the standard daily diet is a reference diet based on the average caloric intake and average protein consumption for the mammalian subject, adjusted for weight, and optionally age or developmental stage, the method further comprises determining the BCAA content in the reference daily diet and reducing the amino acid content in the standard daily diet to provide the reduced BCAA diet.

In an aspect, a reference diet is the diet reported in NHANES III (1988-1994) having a mean of 13.61 grams of BCAAs and a mean of 61.75 grams of non-BCAA, wherein the non-BCAA mean weight does not include asparagine.

When the BCAAs are reduced, the daily intake of BCAAs in the reduced BCAA daily diet provides at least the recommended daily allowance of isoleucine, leucine and valine for the mammal. The recommended daily allowances for a human are approximately 19 mg/kg/day of isoleucine, 42 mg/kg/day of leucine and 4 mg/kg/day of valine for a human adult over the age of 19. The recommended daily allowance of other species varies widely but can be obtained from reference volumes (e.g. for dogs and cats, National Research Council. 2006. Nutrient Requirements of Dogs and Cats. Washington, D.C.: The National Academies Press. https://doi.org/10.17226/10668; for pigs, National Research Council. 2012. Nutrient Requirements of Swine: Eleventh Revised Edition. Washington, D.C.: The National Academies Press. https://doi.org/10.17226/13298); alternatively, nutrient requirements can be determined experimentally. In general, the nutrient requirement for animals such as dogs and cats varies based upon the amount of dry mass of food consumed by the animal, as well as the animal's stage of life. For adult dogs, the recommended daily allowance of BCAAs per kg of body weight is 120 mg/kg/day Ile, 220 mg/kg/day Leu and 160 mg/kg/day Val.

As the level of BCAAs consumed by any subject may vary significantly, calculation of the normal daily intake for each subject, e.g., the standard daily diet, may be personalized, particularly if the degree of restriction for humans exceeds 50%.

The amount of BCAAs normally consumed during the course of a day by a human or animal eating a variety of foods, e.g., the standard diet, may be calculated with the use of a food diary (paper-based or electronic) by a nutritionist, dietician, or similar, or may be calculated by a human (e.g., the subject) with the aid of a computer program (e.g., an “App”); alternatively, the BCAAs normally consumed during the day may be estimated using similar techniques based on the number of calories consumed. The BCAAs normally consumed during the course of a day by an animal eating a single food source (e.g., dog or cat food) may be determined by measuring the mass of the food consumed over the course of a day and calculating the BCAA content with the aid of paper or electronic references or programs containing information about the specific food source being consumed, or about generic diets consumed by the species in question.

The reduced BCAA daily diet includes all essential amino acids.

The percentage of calories from non-BCAAs in the reduced BCAA daily diet are not substantially reduced, e.g., the daily diet may contain sufficient non-BCAAs such that at least 80% of the calories normally consumed by the subject in the form of protein are now provided in the form of protein, BCAAs and/or non BCAAs. This means that the percentage of BCAAs in the diet is reduced relative to the percentage of total amino acids in the diet.

The reduced BCAA daily diet has at least the same number of calories as the standard daily diet for the mammalian subject. Unlike traditional weight loss programs, caloric restriction is not required.

As used herein, the standard daily diet for the mammalian subject is the daily diet consumed by the mammalian subject prior to reduction of BCAAs. In general, a standard daily diet for a human subject is 10-35% of calories from protein, 45-65% of calories from carbohydrate and 20-35% of calories from fat. Most humans, for example, obtain 16-17% of their calories from protein. Humans are generally recommended to consume 10% to 35% of their calories from protein. American men from 20-71+ years, for example, consume 2000-2700 calories per day, with 16-17% of calories from protein, 46-47% of calories from carbohydrates and 33-35% of calories from fat. American women aged 20-71+ years consume 1600-2000 calories per day, with 15-16% calories from protein, 48-50% calories from carbohydrate, and 34-35% calories from fat. On average, according to some studies, men and women >20 years consume 2200 calories per day, with 16% of calories from protein, 48% of calories from carbohydrates, 34% of calories from fat, 3% of calories from alcohol.

In an aspect, the standard daily diet of a mammalian subject comprises excess daily calories for the mammalian subject. In an aspect, excess daily calories means that the weight and BMI of an overweight or obese individual is stable or increasing.

In an aspect, the obese or overweight individual consuming excess calories is eating a relatively healthy and balanced diet containing fruits, vegetables, and the like, but simply is consuming too many calories to reduce their weight.

In an aspect, the standard daily diet for the mammalian subject, specifically a human subject, comprises a standard Western Diet. Western Diets are generally characterized as rich in red meat, dairy products, processed and artificially sweetened foods, and salt, with minimal intake of fruits, vegetables, fish, legumes, and whole grains. The term “standard Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60% or more than or 70% of total calories come from these sources.

In an aspect, the individual consuming a standard Western Diet is also consuming excess daily calories.

In an aspect, consumption of the reduced BCAA daily diet is continued for a period of time sufficient to result in weight loss in the mammalian subject.

In another aspect, consumption of the reduced BCAA daily diet is continued for a period of time sufficient to reduce fat while not substantially decreasing lean muscle mass in the mammalian subject.

In an aspect, consumption of the reduced BCAA daily diet is continued indefinitely.

A reduced BCAA daily diet can be achieved in several different ways.

In an aspect, the reduced BCAA daily diet comprises one, two or three meal replacements, e.g., medical meal replacements per day, the meal replacements containing either no BCAAs, incidental levels of BCAAs from flavoring agents (e.g. fruit juice) used to increase the meal replacement palatability, or simply a reduced level of BCAAs as compared to the average level of BCAAs found in a meal that contains a similar amount of protein for that species. For humans, dietary BCAAs typically consist of approximately 17.2 wt % of dietary protein; thus the percentage of BCAAs in the medical meal replacements is less than 17.2 wt % of the mass of protein plus amino acids.

In general, meals provide the majority of nutrition to a subject. Meals for human subjects can have, for example 300 to 900 calories. Snacks are small and for a human typically have less than 200 calories. Exemplary medical meal replacements include a medical meal replacement beverage, or a solid food product, e.g., a bar, biscuit, wafer or stick which may be scored to provide a particular size. In an aspect, the medical meal replacement comprises BCAD2, which can be formulated so that it is palatable to a human subject without significantly increasing the amount of BCAAs. BCAD2 is a meal replacement that is free of the branched chain amino acids isoleucine, leucine, and valine for the dietary management adults with maple syrup urine disease (MSUD) and related inborn errors of metabolism. The product provides all other essential amino acids as well as nonessential amino acids, carbohydrate, fat, essential fatty acids, vitamins and minerals. The composition of BCAD2 is provided in the Table.

### Composition of BCAD2

| Component | Wt % |
|---|---|
| Protein Equivalent (% Calories)^{†} | 24 |
| Fat (% Calories) | 19 |
| Carbohydrate (% Calories) | 57 |

In an aspect, the medical meal replacement comprises other products suitable for the dietary management of infants, children or adults with MSUD and related inborn errors of metabolism, including but not limited to BCAD1, KETONEX®-1, KETONEX®-2, MSUD Anamix®, MSUD Maxamum®, Complex™ Essential MSD, Complex™ MSD Amino Acid Blend, MSUD Lophlex® LQ, Complex™ Junior MSD w/ DHA-ARA, MSUD Gel™, MSUD Express™, MSUD Cooler®, and CAMINO PRO®MSUD Fruit Punch.

In other aspects, medical meal replacement beverages can be supplied as pre-made drinks, made with a variety of recipes, or as a powder with recipes and instructions for use. In an aspect, a medical meal replacement beverage is in the form of a premixed individual packet that can be combined with water or another beverage as instructed. Medical meal replacement beverages can be supplied as bulk powders, or in individual servings/packets.

In an aspect, a medical meal replacement may be customized for an individual meal plan, that is, a medical meal replacement may vary in both % BCAA restriction and/or assumptions based upon an individual's needs based upon a calculated or estimated standard daily diet.

In another aspect, the reduced BCAA daily diet comprises a low protein daily diet and a supplement comprising essential non-BCAAs, wherein the low protein daily diet has less than 9% of calories derived from protein. Supplements can include free amino acids, salts of amino acids and small peptides such as di- and tri-peptides. The supplement optionally includes non-essential non-BCAAs. The low protein daily diet and/or the supplement can be prescribed by a medical professional. Alternatively, the low protein daily diet can be sold without a prescription as either as an “over-the-counter” pharmacy item or a grocery meal system, available in a range of sizes and recipes suitable for consumption by a wide range of people. The supplement may also be available as an “over-the-counter” pharmacy item. The supplement can be in the form of a pharmaceutical composition, one or more pills, a beverage, or a food item.

As used herein a pharmaceutical composition includes free amino acids, salts of amino acids and small peptides such as di- and tri-peptides together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

In an aspect, all three meals, and optionally snacks, can be replaced by reduced BCAA replacements. The total amount of BCAAs consumed during the daily diet would meet or exceed the RDA for BCAAs, but would be reduced compared to the standard diet.

In another aspect, a method of feeding a mammalian subject in need of weight reduction comprises

reducing the mammalian subject's consumption of BCAAs by the mammalian subject consuming a reduced BCAA meal replacement at least once per day,
wherein the percentage of calories from BCAAs in the reduced BCAA meal replacement are reduced compared to the percentage of calories from BCAAs in a standard meal for the mammalian subject, and
wherein the reduced BCAA meal replacement includes the essential amino acids histidine, lysine, methionine, phenylalanine, threonine, and tryptophan.

In an aspect, the reduced BCAA meal replacement comprise no BCAAs.

In an aspect, the percentage of calories from non-BCAAs in the reduced branched chain amino acid meal replacement are not substantially reduced or increased compared to the percentage of calories from non-BCAAs in the standard meal for the mammalian subject. An individual meal replacement may have reduced non-BCAA levels, with increased levels of non-BCAAs consumed at other meals or through a supplement with increased levels of non-BCAAs.

In another aspect, the inventors have unexpectedly discovered that adding leucine to a low amino acid diet, e.g., a low protein diet, promotes weight loss.

In an embodiment, a method of feeding a mammalian subject in need of weight reduction comprises

the mammalian subject consuming a low protein daily diet supplemented with leucine, wherein the total amount of leucine in the low protein diet supplemented with leucine is greater than or equal to 1.15 wt % of the total calories and/or comprises a daily consumption of 6 g or more of leucine per day,

wherein the low protein diet comprises less than 9% of total calories from protein, and

wherein the normal diet comprises greater than 15% of total calories from protein.

For example, the NHANES III Study suggests the average leucine content of the American diet is 6.08 g/day, with an average protein content of 15.5%. If dietary protein was 17% this would about 6.9 g/day.

The low protein daily diet supplemented with leucine can comprise a low protein daily diet and a supplement comprising leucine. The supplement can be in the form of a pharmaceutical composition, a beverage, or a food item. The low protein daily diet supplemented with leucine can comprise one or more medical meal replacements, such as a medical meal replacement beverage, or a solid food product. The low protein daily diet can be prescribed by a medical professional.

In an aspect, the mammalian subject is a human subject. Exemplary human subjects are overweight, obese, insulin-resistant, have elevated blood glucose levels, pre-diabetes, diabetes, have impaired glucose tolerance, are suffering from fatty liver disease, have cardiovascular disease, or a combination thereof.

In an aspect, the mammalian subject can be a human subject consuming a standard Western diet, a human subject consuming excess daily calories, or both.

In other aspect of the methods described herein, the mammalian subject is a non-human animal. The mammalian subject may be a domesticated animal such as a cat, dog, ferret, gerbil, guinea pig, hamster, minipig, mouse, pig, rabbit, rat, or horse. The domesticated animal may be overweight, obese, have pre-diabetes, have diabetes, or a combination comprising one or more of the foregoing.

The mammalian subject may be an animal housed in a facility for research or public viewing such as an animal research facility, animal sanctuary, or zoo. The animal may be overweight, obese, have pre-diabetes, have diabetes, or a combination comprising one or more of the foregoing.

Dietary requirements vary widely between subject species, developmental stage, and activity level, but the leucine and isoleucine requirements of growing kittens are 9-10 g/kg of diet. A standard canine diet has about 1.8 wt % leucine, 0.9 wt % isoleucine, and 1.1 wt % valine. Minimum recommended amounts of BCAAs for canine are about 0.82 g leucine, 0.46 g isoleucine, and 0.59 g valine.

In another aspect, a method of improving metabolic health in a mammalian subject in need of improvement in metabolic health comprises administering to the mammalian subject a compound that inhibits at least a portion of the gastrointestinal absorption of one or more branched chain amino acids.

In an aspect, the compound is an inhibitor of an L-type amino acid transporter, e.g., LAT1. Specific compounds include 2-aminobicyclo-(2,2,1)-heptane-2-carboxylic acid (BCH); (S)-2-amino-3-(4-((5-amino-2-phenylbenzo[d]oxazol-7-yl)methoxy)-3,5-dichlorophenyl) propanoic acid (JPH203, also called KYT-0353); ESK242; ESK246, or a combination thereof.

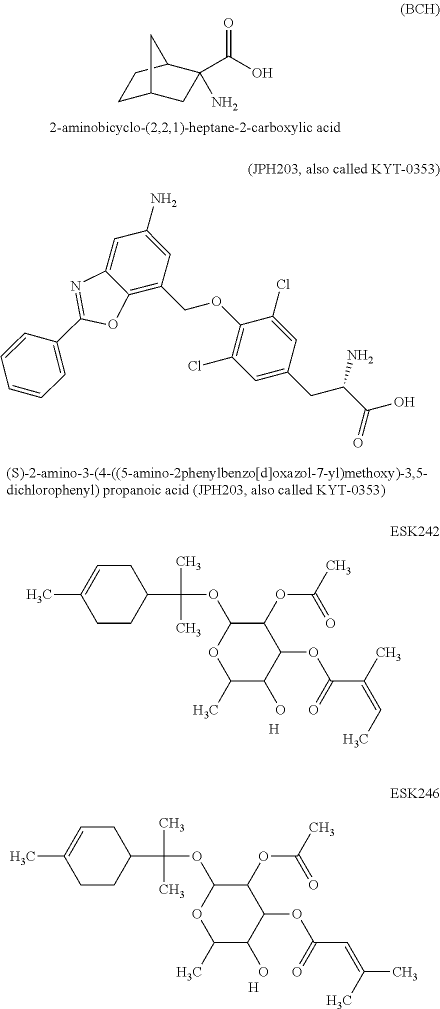

Advantages of the methods disclosed herein include permitting patients to eat to satiety; avoiding major alterations in the consumption of protein, sugar and fat; and particular foods need not be avoided which increases compliance.

The inventors have also unexpectedly found that specially reducing either histidine or reduction of both phenylalanine and tyrosine can also improve metabolic health and reduce weight in mammalian subjects.

A method of improving metabolic health in a mammalian subject in need of improvement in metabolic health comprises

reducing the mammalian subject's consumption either histidine alone or of both phenylalanine and tyrosine by the mammalian subject consuming a reduced amino acid daily diet, wherein the reduced amino acid daily diet has the following characteristics:
a percentage of calories from either histidine alone or of both phenylalanine and tyrosine consumed during the course of the day are reduced compared to a percentage of calories from either histidine alone or of both phenylalanine and tyrosine in a standard daily diet for the mammalian subject,
the daily intake of either histidine alone or of both phenylalanine and tyrosine in the reduced amino acid daily diet provides at least the recommended daily allowance of histidine, phenylalanine and tyrosine for the mammalian subject
the reduced amino acid daily diet includes all essential amino acids, and optionally non-essential amino acids,
the percentage of calories from amino acids other than histidine or phenylalanine and tyrosine in the reduced BCAA daily diet are not substantially reduced compared to their percentages in the standard daily diet for the mammalian subject, and
the reduced amino acid daily diet has at least the same number of calories as the standard daily diet for the mammalian subject.

Exemplary subjects include humans and other mammals as described herein.

In yet another aspect, a method of feeding a mammalian subject in need of weight reduction comprises

reducing the mammalian subject's consumption of either histidine alone or of both phenylalanine and tyrosine by the mammalian subject consuming a reduced amino acid meal replacement at least once per day, wherein the percentage of calories from histidine or of both phenylalanine and tyrosine in the reduced amino acid meal replacement are reduced compared to the percentage of calories from histidine alone or of both phenylalanine and tyrosine in a standard meal for the mammalian subject, and wherein the reduced amino acid meal replacement includes all essential amino acids.

Exemplary reductions include at least 25%, 40%, 50%, 67% or more compared to the percentage of calories from either histidine alone or of both phenylalanine and tyrosine in a standard daily diet for the mammalian subject. If both phenylalanine and tyrosine are reduced, the reduction in phenylalanine may be greater than or equivalent to the percentage reduction in tyrosine.

The recommended daily allowances for a human are approximately 14 mg/kg/day of histidine, and 33 mg/kg/day of phenylalanine and tyrosine combined for a human adult over the age of 19. The recommended daily allowance of other species varies widely but can be obtained from reference volumes (e.g. for dogs and cats, National Research Council. 2006. Nutrient Requirements of Dogs and Cats. Washington, D.C.: The National Academies Press. https://doi.org/10.17226/10668; for pigs, National Research Council. 2012. Nutrient Requirements of Swine: Eleventh Revised Edition. Washington, D.C.: The National Academies Press. https://doi.org/10.17226/13298); alternatively, nutrient requirements can be determined experimentally. In general, the nutrient requirement for animals such as dogs and cats varies based upon the amount of dry mass of food consumed by the animal, as well as the animal's stage of life. For adult dogs, the recommended daily allowance of amino acids per kg of body weight is estimated to be approximately 22 mg/kg/day histidine, and 85.8 mg/kg/day of phenylalanine and tyrosine combined.

In an embodiment, reduced dietary levels of either histidine alone, or of both phenylalanine and tyrosine, or of all three amino acids are achieved in whole or in part through the consumption of foods containing glycomacropeptide (GMP).

GMP is a 64 AA glycosylated peptide that occurs naturally in bovine milk within the whey fraction and is released in the newborn and adult human gastrointestinal tract by pepsin mediated proteolysis after milk ingestion. Commercial GMP is a by-product of cheese production when bovine κ-casein is cleaved by the action of chymosin into para-κ-casein, which remains with the curd, and κ-caseino glycomacropeptide or GMP, which remains in the whey. GMP is an abundant protein as it comprises 15 to 20% of the total protein in sweet cheese whey. GMP protein contains 47% (w/w) indispensable amino acids, but contains no histidine (His), tryptophan (Trp), tyrosine (Tyr), arginine (Arg), Cysteine (Cys) or Phe. U.S. Pat. No. 8,604,168 describes medical foods containing GMP for the management of phenylketonuria.

For specific reduction of histidine, phenylalanine and tyrosine would also be supplemented up such that the mammalian subject would consume approximately normal levels of phenylalanine and tyrosine. For specific reduction of phenylalanine and tyrosine, histidine would be also be supplemented up such that the mammalian subject would consume approximately normal levels of histidine.

In yet another aspect, mammalian subjects would be provided with pre-packaged foods and/or beverages with naturally-derived proteins other than GMP that contain lower than average levels of histidine or lower than average levels of both phenylalanine and tyrosine. Subjects consuming a daily diet primarily consisting of the prepackaged foods and/or beverages would have a reduced consumption of histidine or a reduced consumption of both phenylalanine and tyrosine.

In another aspect, the reduced amino acid meal replacement or the reduced amino acid daily diet contain natural proteins with reduced levels of specific amino acids but does not contain glycomacropeptide.

The invention is further illustrated by the following non-limiting examples.

### EXAMPLES

### Methods

### Experimental Procedures for Examples

Animals and Diets: Male C57BL/6J mice were purchased from The Jackson Laboratory at 5 weeks of age, and pre-conditioned with Western diet (TD.88137, Envigo, Madison, Wis.) starting at 6 weeks of age for 12 weeks; chow control mice were fed Purina 5001. Amino acid defined diets as well as Western diet supplemented with additional BCAAs were also obtained from Envigo. Amino acid defined diet compositions and item numbers are provided in Tables 1 and 2.

TABLE 1

| | Amino Acid Defined Diets | | | | |
|---|---|---|---|---|---|
| | Control | ExLow | ExLow | Western | WD + |
| | AA | AA | BCAA | (WD) | BCAA |
| | Teklad Diet number | | | | |
| | | TD. | | | TD. |
| | TD. 140711 | 140918 | TD. 150387 | TD. 88137 | 150386 |
| | Color | | | | |
| | Red | Orange | Blue | Tan | Green |
| Formula | g/kg | g/kg | g/kg | g/kg | g/kg |
| L-Alanine | 9.38 | 2.18 | 12.1566 | — | — |
| L-Arginine | 6.3 | 1.46 | 6.3 | — | — |
| L-Asparagine | 20.58 | 4.79 | 22.6388 | — | — |
| L-Aspartic Acid | 20.58 | 4.79 | 24.729 | — | — |
| L-Cysteine | 7.2 | 1.67 | 7.2 | — | — |
| L-Glutamic Acid | 28.97 | 6.74 | 33.5548 | — | — |
| L-Glutamine | 33.77 | 7.87 | 36.0672 | — | — |
| Glycine | 2.96 | 0.69 | 5.2991 | — | — |
| L-Histidine HCl, | 4.6 | 1.07 | 4.6 | — | — |
| monohydrate | | | | | |
| L-Isoleucine | 7.8 | 1.81 | 1.81 | — | 8.8725 |
| L-Leucine | 25.4 | 5.9 | 5.9 | — | 15.6195 |
| L-Lysine HCl | 20.38 | 4.74 | 20.38 | — | — |
| L-Methionine | 6.7 | 1.56 | 6.7 | — | — |
| L-Phenylalanine | 6.6 | 1.54 | 6.6 | — | — |
| L-Proline | 7.41 | 1.72 | 10.9965 | — | — |
| L-Serine | 7.41 | 1.72 | 10.6844 | — | — |
| L-Threonine | 9.7 | 2.26 | 9.7 | — | — |
| L-Tryptophan | 3.4 | 0.79 | 3.4 | — | — |
| L-Tyrosine | 6.9 | 1.61 | 6.9 | — | — |
| L-Valine | 8.4 | 1.95 | 1.95 | — | 10.725 |
| DL-Methionine | — | — | — | 3.0 | 3.0 |
| Casein | — | — | — | 195 | 195 |
| Sucrose | 291.248 | 291.248 | 291.248 | 341.46 | 341.46 |
| Corn Starch | 150.0 | 243.79 | 153.4368 | 150 | 114.683 |
| Maltodextrin | 150.0 | 243.79 | 153.4368 | — | — |
| Anhydrous Milkfat | — | — | — | 210 | 210 |
| Cholesterol | — | — | — | 1.5 | 1.5 |
| Corn Oil | 52.0 | 52.0 | 52.0 | — | — |
| Olive Oil | 29.0 | 29.0 | 29.0 | — | — |
| Cellulose | 30.0 | 30 | 30 | 50 | 50 |
| Mineral Mix, AIN-93M-MX | 35.0 | 35 | 35 | — | — |
| (94049) | | | | | |
| Mineral Mix, AIN-76 | — | — | — | 35 | 35 |
| (170915) | | | | | |
| Calcium Carbonate | — | — | — | 4 | 4 |
| Calcium Phosphate Ca(H₂PO₄)₂•H2O | 8.2 | 8.2 | 8.2 | — | — |
| Vitamin Mix, Teklad (40060) | 10.0 | 10.0 | 10.0 | 10.0 | 10.0 |
| TBHQ, antioxidant | 0.012 | 0.012 | 0.012 | — | — |
| Ethoxyquin, antioxidant | — | — | — | 0.04 | 0.04 |
| Food Coloring | 0.1 | 0.1 | 0.1 | 0.1 | 0.1 |
| % kcal from | | | | | |
| Protein (based on N × 6.25) | 22 | 5.1 | 21.9 | 15.2 | 18.3 |
| Carbohydrates | 59.4 | 76.4 | 59.6 | 42.7 | 39.8 |
| Fat | 18.6 | 18.5 | 18.5 | 42 | 41.9 |
| Kcal/g | 3.9 | 3.9 | 3.9 | 4.5 | 4.6 |

TABLE 2

| Formula | g/kg | g/kg | g/kg | g/kg | g/kg |
|---|---|---|---|---|---|
| L-Alanine | 9.38 | 9.38 | 5.5861 | 11.8183 | 3.05 |
| L-Arginine | 6.3 | 6.3 | 6.3 | 6.3 | 2.05 |
| L-Asparagine | 20.58 | 20.58 | 17.7668 | 22.388 | 6.7 |
| L-Aspartic Acid | 20.58 | 20.58 | 14.9108 | 24.2237 | 6.7 |
| L-Cysteine | 7.2 | 7.2 | 7.2 | 7.2 | 2.34 |
| L-Glutamic Acid | 28.97 | 28.97 | 22.7053 | 32.9963 | 9.43 |
| L-Glutamine | 33.77 | 33.77 | 30.6311 | 35.7873 | 11.0 |
| Glycine 2.96 | 2.96 | 2.96 | 0.96 | 5.0141 | 0.96 |
| L-Histidine HCl, monohydrate | 4.6 | 4.6 | 4.6 | 4.6 | 1.5 |
| L-Isoleucine | 7.8 | 7.8 | 15.6 | 2.54 | 2.54 |
| L-Leucine | 25.4 | 25.4 | 50.8 | 8.27 | 8.27 |
| L-Lysine HCl | 20.38 | 20.38 | 20.38 | 20.38 | 6.64 |
| L-Methionine | 6.7 | 6.7 | 6.7 | 6.7 | 2.18 |
| L-Phenylalanine | 6.6 | 6.6 | 6.6 | 6.6 | 2.15 |
| L-Proline | 7.41 | 7.41 | 2.5094 | 10.5596 | 2.41 |
| L-Serine | 7.41 | 7.41 | 2.9359 | 10.2855 | 2.41 |
| L-Threonine | 9.7 | 9.7 | 9.7 | 9.7 | 3.16 |
| L-Tryptophan | 3.4 | 3.4 | 3.4 | 3.4 | 1.1 |
| L-Tyrosine | 6.9 | 6.9 | 6.9 | 6.9 | 2.25 |
| L-Valine | 8.4 | 8.4 | 16.8 | 2.735 | 2.735 |
| Sucrose | 291.248 | 341.46 | 341.46 | 341.46 | 341.46 |
| Corn Starch | 150.0 | 49.63 | 45.3573 | 52.6511 | 132.0625 |
| Maltodextrin | 150.0 | 49.63 | 45.3573 | 52.6511 | 132.0625 |
| Anhydrous Milkfat | — | 210 | 210 | 210 | 210 |
| Cholesterol | — | 1.5 | 1.5 | 1.5 | 1.5 |
| Corn Oil | 52.0 | — | — | — | — |
| Olive Oil | 29.0 | — | — | — | — |
| Cellulose | 30.0 | 50 | 50 | 50 | 50 |
| Mineral Mix, AIN-93M-MX (94049) | 35.0 | 35 | 35 | 35 | 35 |
| Calcium Phosphate Ca(H₂PO₄)₂•H2O | 8.2 | 8.2 | 8.2 | 8.2 | 8.2 |
| Vitamin Mix, Teklad (40060) | 10.0 | 10.0 | 10.0 | 10.0 | 10.0 |
| TBHQ, antioxidant | 0.012 | 0.04 | 0.04 | 0.04 | 0.04 |
| Food Coloring | 0.1 | 0.1 | 0.1 | 0.1 | 0.1 |
| % kcal from | | | | | |
| Protein (based on N × 6.25) | 22 | 20.7 | 21.4 | 20.2 | 6.8 |
| Carbohydrates | 59.4 | 38.5 | 37.8 | 39.0 | 52 |
| Fat | 18.6 | 40.9 | 40.8 | 40.9 | 41.2 |
| Kcal/g | 3.9 | 4.6 | 4.6 | 4.6 | 4.6 |

Procedures:

Glucose tolerance tests were performed by fasting the mice overnight for 16 hours and then injecting glucose (1 g/kg) intraperitoneally as described in the art. Insulin tolerance tests were performed by fasting mice for 4 hours starting at lights on, and then injecting insulin (0.75 U/kg) intraperitoneally. Glucose measurements were taken using a Bayer Contour blood glucose meter and test strips. Blood for determination of fasting glucose and insulin was obtained following an overnight fast; insulin levels were determined by ELISA (Crystal Chem). Mouse body composition was determined using an EchoMRI 3-in-1 Body Composition Analyzer (Houston, Tex.) according to the manufacturer's procedures. For assay of multiple metabolic parameters (O2, CO2, food consumption) and activity tracking, mice were acclimated to housing in a Columbus Instruments Oxymax/CLAMS metabolic chamber system (Columbus, Ohio) for approximately 24 hours, and data from a continuous 24 hour period was then recorded and analyzed. FGF21 levels were determined by ELISA (R&D Systems) using plasma collected from mice sacrificed between 8 am and 12 pm after an approximately 16 hour fast, while tissues for molecular analysis were flash-frozen in liquid nitrogen and prepared.

Histology:

Samples of brown adipose tissue, liver, and skin were isolated from mice on the indicated diets following euthanasia. Sections of brown adipose tissue were fixed in 4% paraformaldehyde overnight, sectioned and H&E stained by the UWCCC Experimental Pathology Laboratory. Sections of liver were embedded in OCT, and then cryosectioned and Oil-Red-O stained by the UWCCC Experimental Pathology Laboratory. Samples of skin were isolated from the belly and back of mice on the indicated diets, and sections were paraformaldehyde-fixed (4%) overnight and then paraffin-embedded for evaluation as described in the art. For quantification of lipid droplet size in brown adipose tissue and liver, 6 independent fields were obtained for tissue from each mouse and quantified using NIH ImageJ.

Quantitative PCR:

Total liver or adipose RNA was extracted with TRI Reagent® (Sigma). The concentration and purity of RNA were determined by absorbance at 260/280 nm, and 1 μg of RNA was used to generate cDNA (Superscript® III; Invitrogen, Carlsbad, Calif., USA). Oligo dT primers and primers for real-time PCR were obtained from Integrated DNA Technologies (Coralville, Iowa, USA). Reactions were run on an Applied Biosystems StepOne™ Plus machine (Applied Biosystems, Foster City, Calif., USA) with Sybr® Green PCR Master Mix (Invitrogen). Actin was used to normalize the results from gene-specific reactions. Primer sequences are given in Supplemental Table 3.

TABLE 3

| | | SEQ | | SEQ |
|---|---|---|---|---|
| | | ID | | ID |
| | Forward | NO: | Reverse | NO: |
| Acc1 | AAGGCTATGTGAAGGATG | 1 | CTGTCTGAAGAGGTTAGG | 13 |
| Acl | GCCAGCGGGAGCACATC | 2 | CTTTGCAGGTGCCACTTCATC | 14 |
| Actb | ACCTTCTACAATGAGCTGCG | 3 | ACCTTCTACAATGAGCTGCG | 15 |
| Bmp8 | TCAACACAACCCTCCACATCA | 4 | AGATCGGAGCGTCTGAAGATC | 16 |
| Dgat1 | TGGTGTGTGGTGATGCTGATC | 5 | GCCAGGCGCTTCTCAA | 17 |
| Dgat2 | AGTGGCAATGCTATCATCATCAT | 6 | TCTTCTGGACCCATCGGCCCCAGGA | 18 |
| Fasn | CCCCTCTGTTAATTGGCTCC | 7 | TTGTGGAAGTGCAGGTTAGG | 19 |
| Fgf21 | CAAATCCTGGGTGTCAAAGC | 8 | CATGGGCTTCAGACTGGTAC | 20 |
| Gpat | CAACACCATCCCCGACATC | 9 | GTGACCTTCGATTATGCGATCA | 21 |
| Pparg | GTACTGCCGTTTTCACAAGTG | 10 | TCTTTCAGGTCGTGTTCACAG | 22 |
| Scd1 | CTGACCTGAAAGCCGAGAAG | 11 | AGAAGGTGCTAACGAACAGG | 23 |
| Srebp1c | GGAGCCATGGATTGCACATT | 12 | GGCCCGGGAAGTCACTGT | 24 |

Statistics:

Statistical analysis was conducted using Prism 7 (GraphPad Software). Glucose and insulin tolerance tests, as well as other tests involving repeated measurements, were analyzed with two-way repeated-measures ANOVA, followed by a Tukey-Kramer or Dunnett's post-hoc test as specified. All other comparisons of three or more means were analyzed by one-way ANOVA followed by a Dunnett's or Tukey-Kramer post-hoc test as specified. Additional comparisons, if any, were corrected for multiple comparisons using the Bonferroni method.

Islet Isolation and Ex Vivo Studies:

Islets were isolated and an ex vivo glucose stimulated insulin secretion assay was performed as described in the art. Isolated islets were transferred to a 96-well v-bottom plate and incubated with RPMI 1640 medium with 10% FBS, penicillin/streptomycin, and 11.1 mM glucose for 48 hours. Briefly, following a 48-hour incubation period, islets were treated with a low glucose (1.7 mM) Krebs-Ringer bicarbonate buffer pre-incubation solution for 45-minutes followed by stimulatory high glucose (16.7 mM) Krebs-Ringer bicarbonate buffer solution for 45-minutes. Secretory media was then saved and islets were lysed with a Cell Signaling Technologies lysis buffer (9803). Insulin secretion and content were analyzed by ELISA. Mitochondrial membrane potential was measured in islets pre-loaded with Rhodamine123 (5 μM, 5 min) (Sigma) and perfused with a standard external solution (135 mM NaCl, 4.8 mM KCl, 5 mM CaCl₂), 1.2 mM MgCl₂, 20 mM HEPES; pH 7.35) containing 2 or 20 mM glucose, followed by a reference solution containing 20 mM glucose and 5 mM KCN, used to normalize the data. Excitation (500/20×) and emission (535/30 m) filters (ET type, Chroma Technology Corporation) were used in combination with an FF444/521/608-Di01 dichroic (Semrock) on a Nikon Ti-Eclipse microscope. A single region of interest was used to quantify the average response of each islet using Nikon Elements.

### Example 1: Diet-Induced Obese Mice Switched to Normal Calorie Diets with Reduced Branched-Chain Amino Acids Rapidly Lose Weight and Improve Glycemic Control

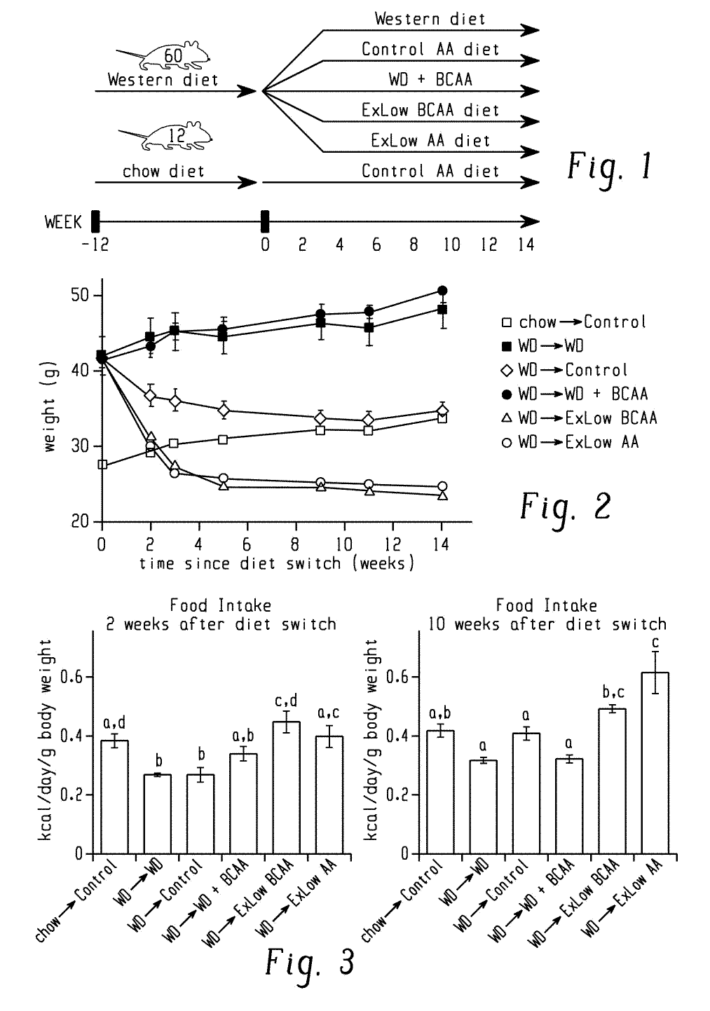

Obesity and metabolic dysfunction were induced by feeding C57BL/6J mice a Western diet for 12 weeks (DIO mice). DIO mice were then switched to one of several different diets of varying amino acid compositions, with an energy density and macronutrient composition typical of rodent chow (FIG. 1). One group of mice was maintained on a Western diet, while an additional group of mice was fed a Western diet with supplemental branched-chain amino acids (BCAAs), which in rats promotes insulin resistance. Finally, a parallel group of mice never exposed to a Western diet was switched to the Control amino acid defined diet. The exact formulations of each diet are provided in Table 1.

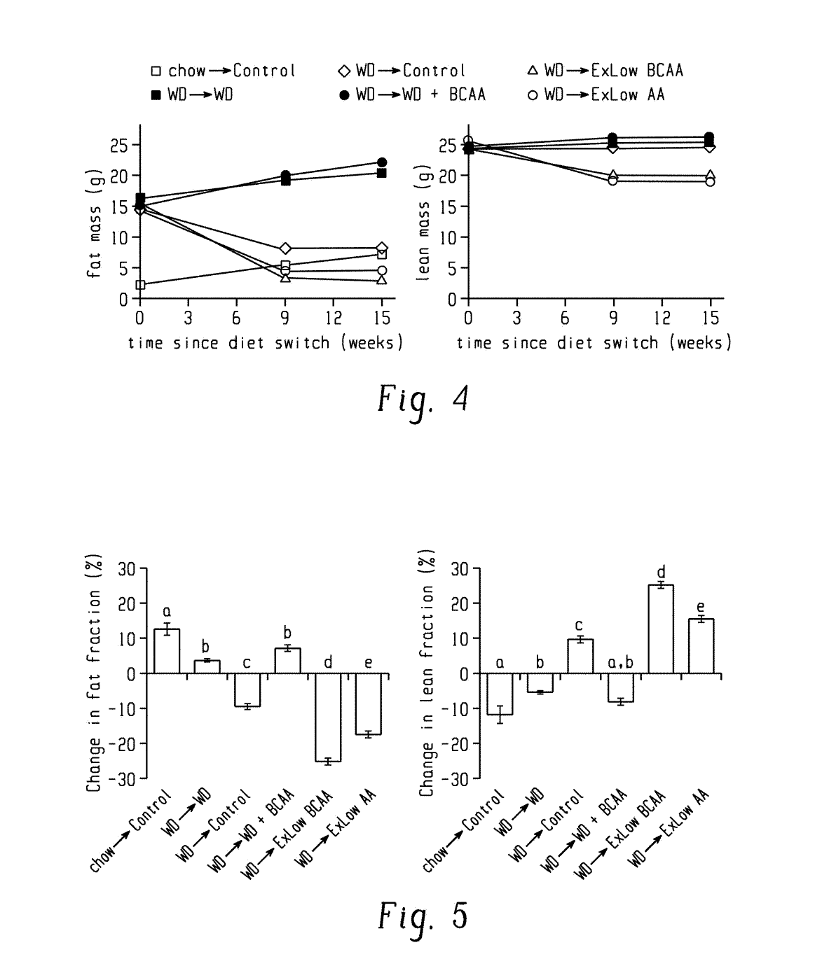

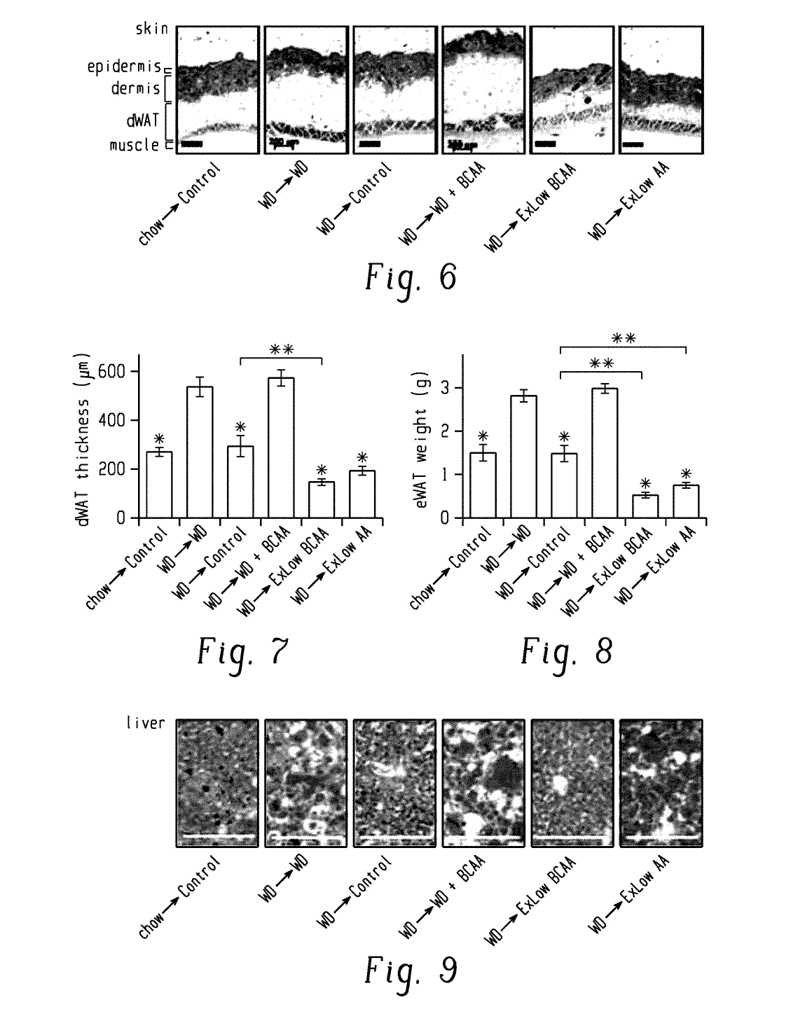

All of the DIO mice lost weight when switched to a normal calorie diet, while mice continuing to consume a Western diet (with or without supplemental BCAAs) continued to gain weight (FIG. 2). Mice consuming diets in which the BCAAs (ExLow BCAA) or all amino acids (ExLow AA) were specifically reduced lost weight very rapidly, normalizing their weight to that of mice never exposed to a WD by shedding approximately 25% of their body weight in two weeks before eventually stabilizing at a lower weight than mice never exposed to a Western diet. In contrast, DIO mice switched to the normal calorie Control diet normalized their weight more slowly, taking two additional months to reach the same weight as mice never exposed to a WD. The weight loss was not due to decreased food consumption, as mice placed on the ExLow BCAA or ExLow AA diets actually consumed more calories per gram of body weight (FIG. 3). While fat mass was significantly reduced, mice placed on the ExLow BCAA or ExLow AA diets also lost some lean mass (FIG. 4); the net effect of these diets was greatly reduced adiposity (FIG. 5). DIO mice switched to any of the normal calorie diets had significantly thinner dermal white adipose tissue (dWAT) thickness (FIG. 6, 7) and decreased epididymal white adipose tissue (eWAT) mass (FIG. 8) relative to mice that remained on a Western diet. Notably, DIO mice fed an ExLow BCAA diet had thinner dWAT and less eWAT than DIO mice fed the Control diet (FIG. 7, 8).

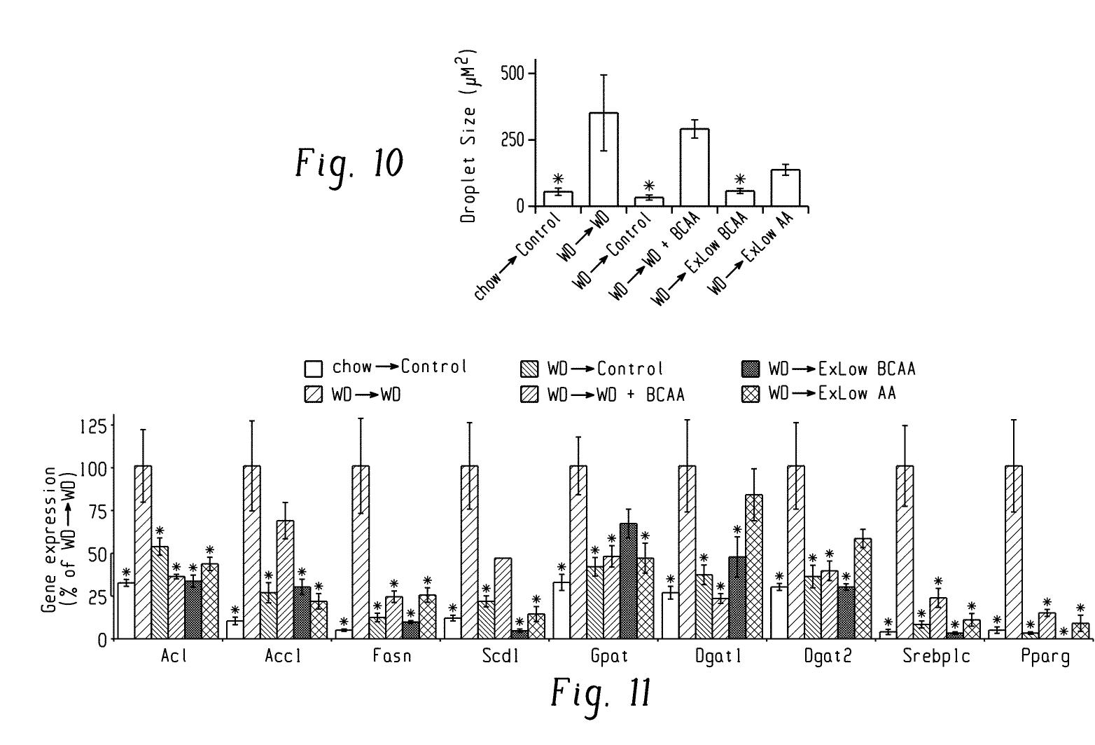

DIO mice that continued to consume Western diet or Western diet supplemented with BCAAs had evident hepatic steatosis with large fat droplets, but DIO mice switched to either the Control or ExLow BCAA diets had decreased liver droplet size and reverted to normal liver histology by the conclusion of the experiment (FIG. 9, 10). DIO mice switched to the ExLow AA diet had a trend towards reduced lipid droplet size (corrected p=0.12) and hepatic steatosis was still evident. Decreased mRNA expression of many lipogenic genes and transcription factors was observed in the livers of mice switched to Control or ExLow BCAA diets, with a similar although less dramatic effect in mice switched to the ExLow AA diet (FIG. 11). Surprisingly, mice fed Western diet supplemented with BCAAs also had decreased hepatic expression of many lipogenic genes and transcription factors.

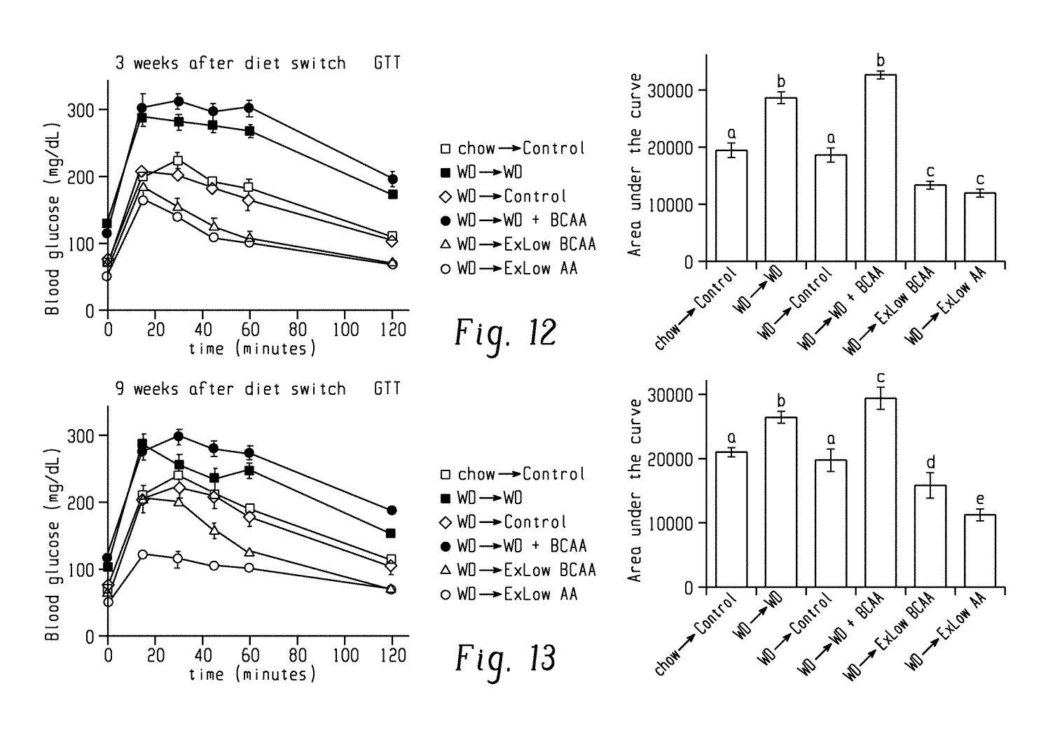

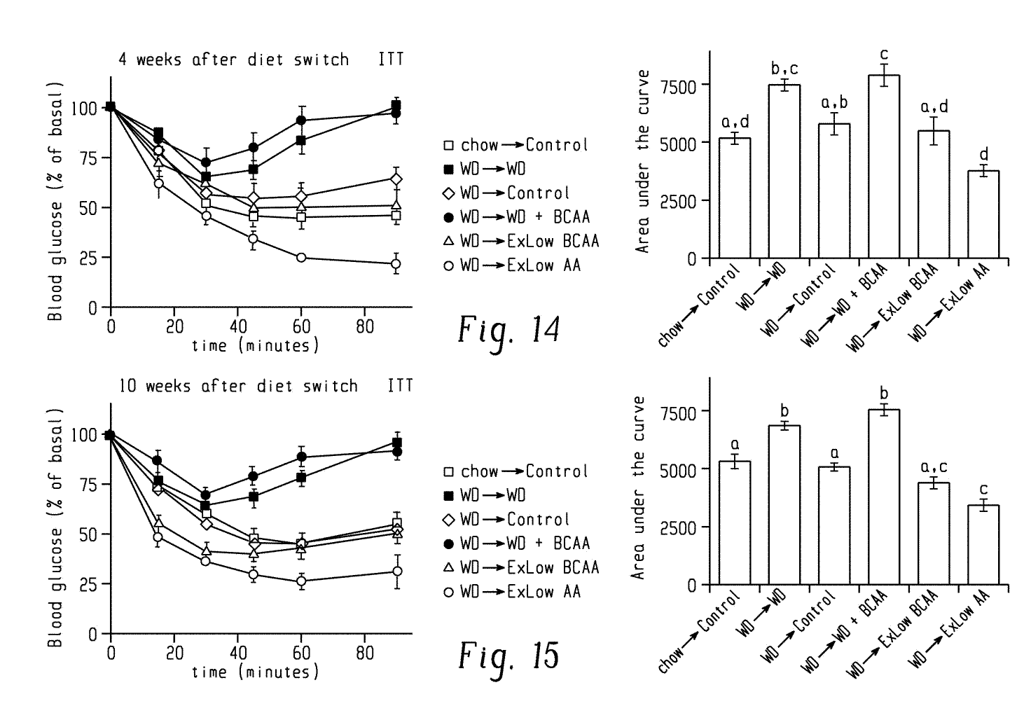

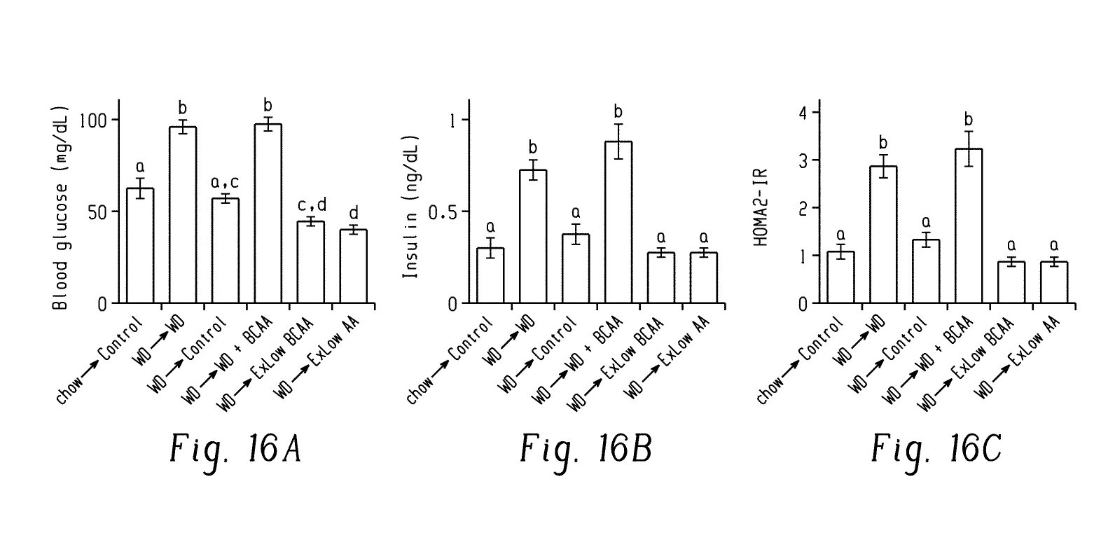

Glucose tolerance was significantly improved in all mice switched to normal calorie diets; after three weeks, mice switched to ExLow BCAA and ExLow AA diets had improved glucose tolerance relative to other groups, including mice never exposed to a Western diet (FIG. 12), and this effect was sustained throughout the experiment (FIG. 13). In contrast, supplementing a Western diet with BCAAs resulted in worse glucose tolerance vs. all other groups after 9 weeks (FIG. 13). DIO mice that remained on a Western diet were insulin resistant; we observed that all mice placed on normal calorie diets showed improved insulin sensitivity relative to mice remaining on a Western diet, with mice switched to an ExLow AA diet showing improved insulin sensitivity relative to all groups (FIG. 14, 15). DIO mice that remained on a Western diet had fasting hyperglycemia and hyperinsulinemia, as well as increased HOMA2-IR, relative to mice never exposed to Western diet (FIG. 16). In agreement with our glucose and insulin tolerance tests, these deficits were corrected in mice switched to any of the normal calorie diets (FIG. 16).

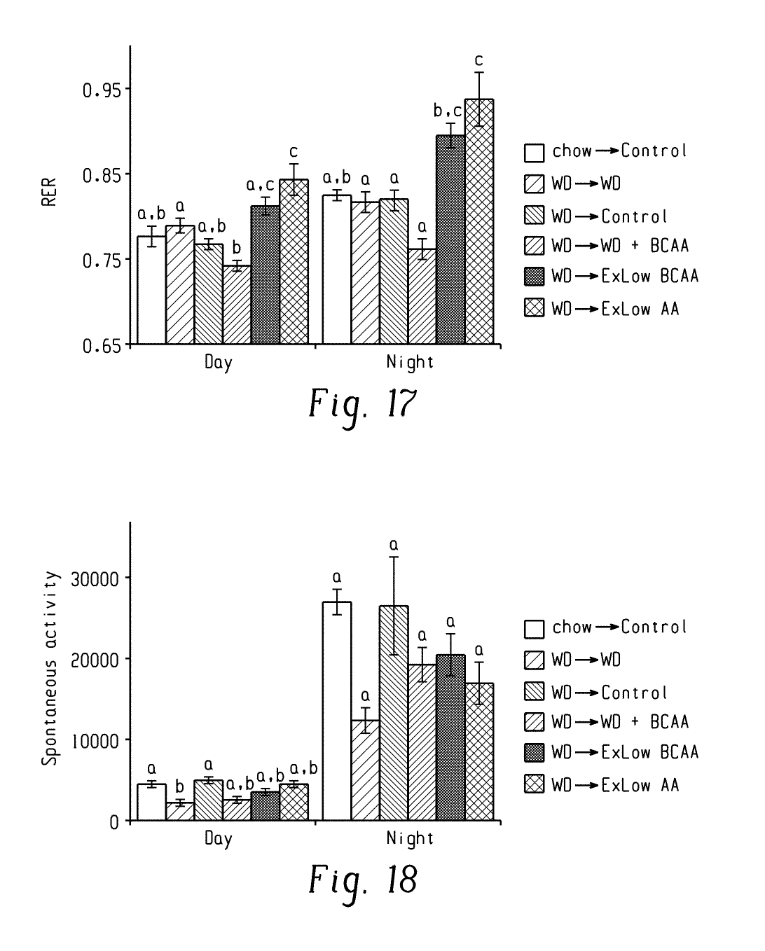

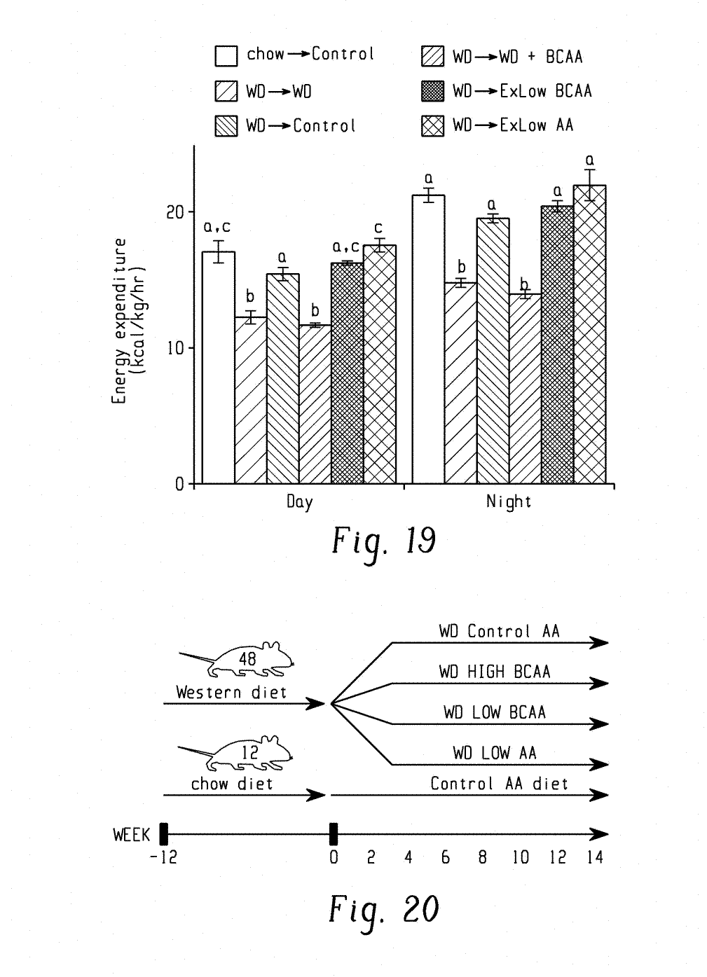

It has been reported that low protein and low amino acid diets promote energy expenditure. Metabolic chambers were used to assess activity and energy expenditure via indirect calorimetry once the weights of all groups had stabilized. As expected, DIO mice switched to the ExLow AA diet, which is high in carbohydrates, had increased respiratory exchange ratio (RER) and increased energy expenditure compared to mice switched to the Control diet; this was not associated with increased activity (FIG. 17-19). Intriguingly, mice consuming the ExLow BCAA diet also had increased RER (FIG. 17); however, mice on the ExLow BCAA diet had similar activity and energy expenditure as other mice consuming a normal calorie diet (FIG. 18, 19).

### Example 2: Specific Restriction of Dietary Branched-Chain Amino Acids Promotes Leanness and Glucose Homeostasis in Diet-Induced Obese Mice Continuing to Consume Western Diet

These results suggested that reducing dietary BCAAs might be an effective means to restore metabolic health; however, the simultaneous alterations in energy density and macronutrient ratios, and the similar though slower changes in mice switched to a normal calorie Control diet made it difficult to elucidate the precise contribution of the BCAAs. Further, mice switched to the ExLow AA or ExLow BCAA diets had reduced adiposity but an absolute loss of lean mass in mice, which may be undesirable. Finally, this type of extreme dietary change might have poor compliance in humans.

To address these shortcomings and specifically determine if altering dietary BCAAs can restore metabolic health to DIO mice, we designed a new series of interventions based on a new amino acid-defined Western diet (WD Control AA), which closely matches the macronutrient profile of the natural sourced WD TD.88137. Using this WD Control AA diet as our base, we developed several additional isocaloric Western diets with increased or decreased dietary levels of BCAAs (Table 4). As shown in FIG. 20, we induced obesity in 48 C57Bl/6J mice by feeding them a Western diet for twelve weeks; these mice were then randomized into four groups of twelve mice each, and each group was placed on either a WD Control AA, WD High BCAA, WD Low BCAA, or WD Low AA diet. In parallel, a group of 12 mice never exposed to a Western diet was switched to the Control amino acid defined diet.

TABLE 4

| Diet Name | Description |
|---|---|
| WD Control AA | AA-defined “Western” diet - 41% of calories from |
| | fat, 38% of calories from carbohydrates (high |
| | sucrose), with cholesterol |
| WD High BCAA | Similar to WD Control AA; with 2x BCAAs |
| WD Low BCAA | Similar to WD Control AA; with 67% reduction in |
| | the BCAAs |
| WD Low AA | Similar to WD Control AA; with 67% reduction in |
| | all AAs |
| Control AA | AA defined normal calorie diet |

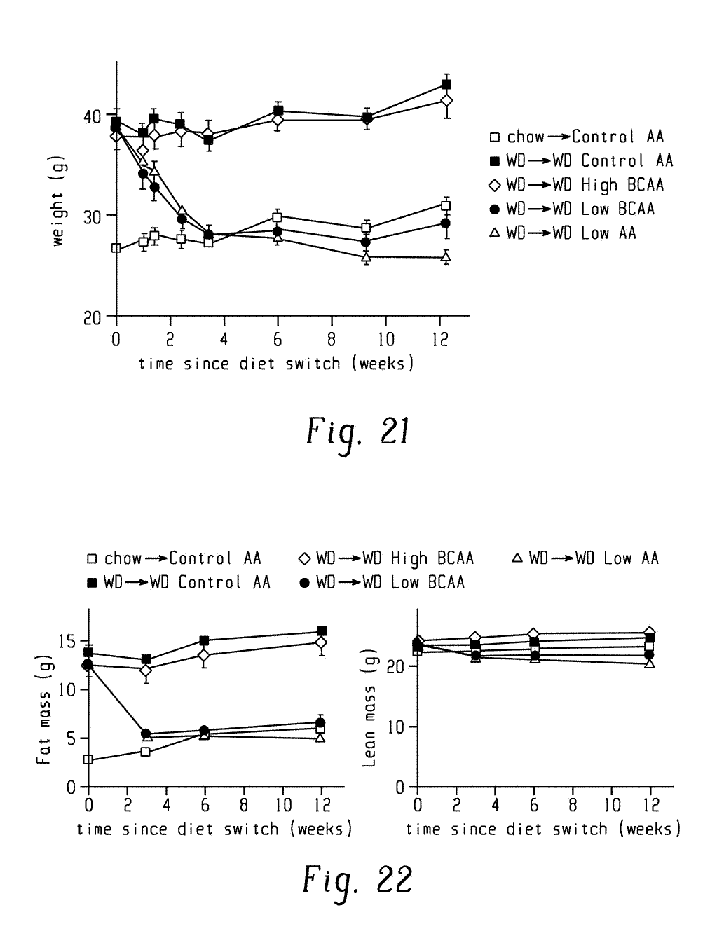

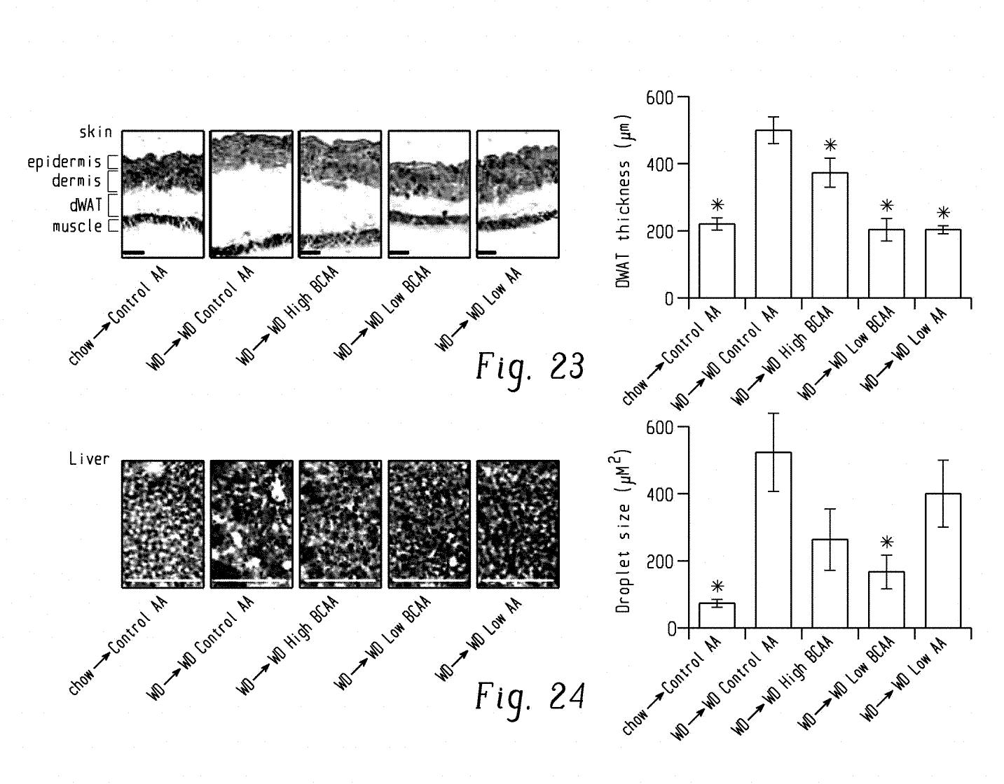

While DIO mice fed either the WD Control AA or WD High BCAA diets maintained or gained weight, DIO mice fed either the WD Low BCAA diet or the WD Low AA diet progressively lost weight over the first three weeks (FIG. 21). The weight of mice on a WD Low BCAA diet then stabilized, matching the weight of mice fed a Control AA diet never been exposed to a Western diet; mice fed a WD Low AA diet continued to lose weight, albeit at a reduced weight, over the subsequent nine weeks. The weight loss of mice on these two diets was due to a dramatic decrease in fat mass, while lean mass was preserved (FIG. 22). One consequence of this change was a dramatic decrease in dWAT thickness in mice fed the WD Low BCAA or WD Low AA diets relative to mice on the WD Control AA diet; curiously, mice fed the WD High BCAA diet also had decreased dWAT thickness despite not losing adipose mass (FIG. 23).

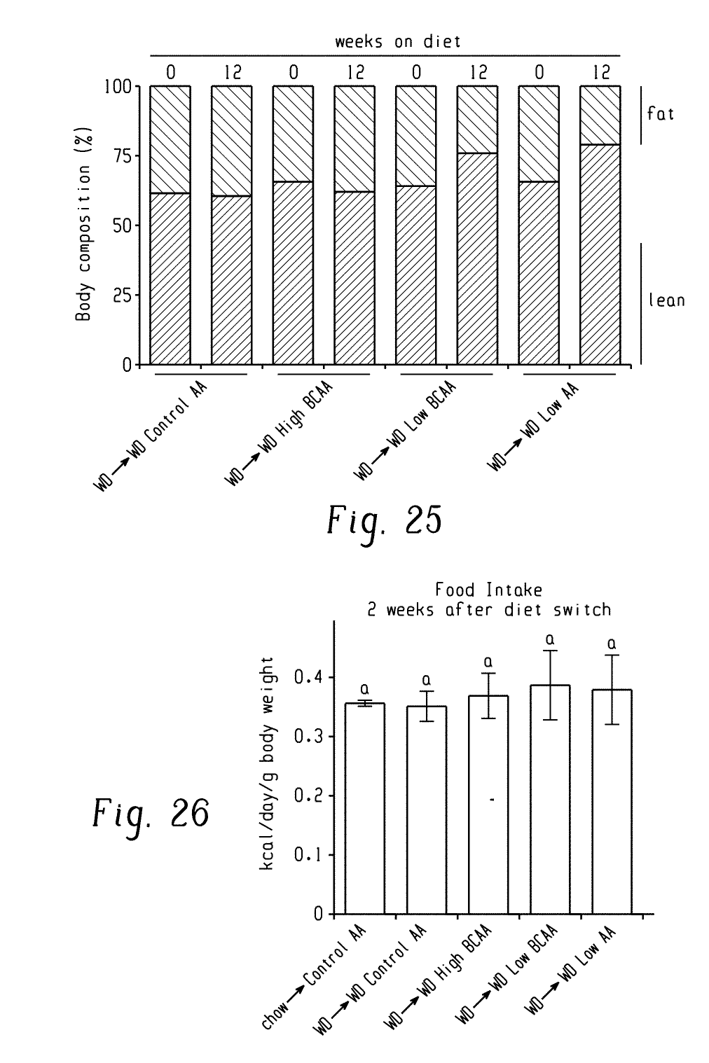

Mice fed the WD Low BCAA diet had significantly smaller hepatic lipid droplets than mice fed the WD Control AA diet, and appeared to have cleared much of the hepatic fat deposited by Western diet feeding (FIG. 24). Intriguingly, there was also some histological improvement and a trend towards reduced liver droplet size in mice consuming extra BCAAs (WD High BCAA). Mice fed the WD Low AA diet did not have smaller lipid droplets and appeared to still have significant fat content (FIG. 24), even though mice on this diet also had dramatic reductions in adipose mass and dWAT thickness. Overall, mice fed the WD Low BCAA or WD Low AA diets showed significant improvement in body composition, with decreased adiposity and a resulting increase in the lean fraction (FIG. 25). This weight loss was not the results of decreased food consumption, as mice fed WD Low BCAA and WD Low AA diets ate the same number of calories as mice on a WD Control AA diet (FIG. 26). Notably, increasing dietary BCAAs (WD High BCAA) had no significant effect on weight, body composition, or food intake (FIG. 21-24).

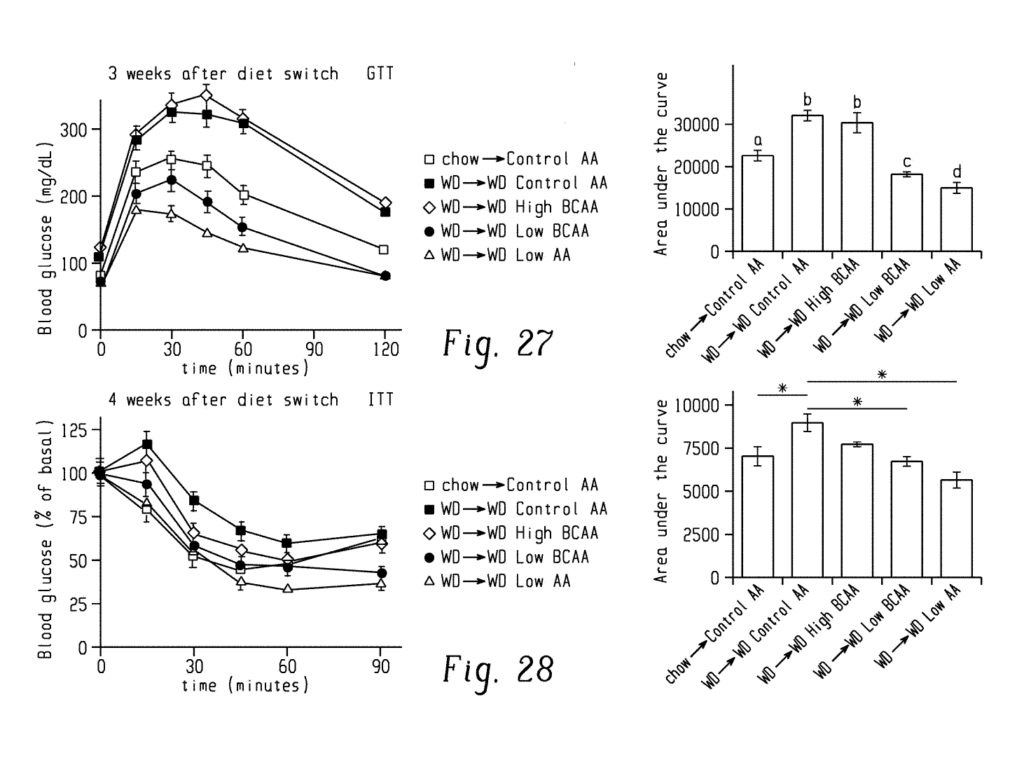

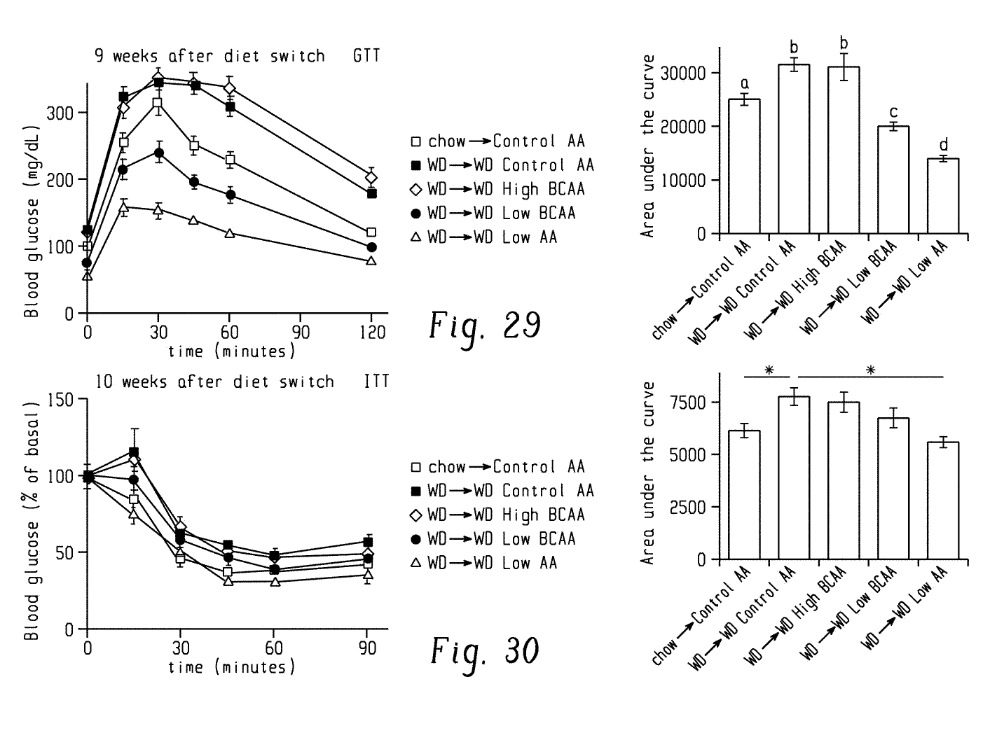

Over the course of this study, glycemic control was examined by conducting glucose and insulin tolerance tests, and by collecting blood to determine fasting glucose and insulin levels. Increased glucose tolerance was observed in mice eating the WD Low BCAA and WD Low AA diets at 3 weeks after diet switch—a time point at which these mice still weighed more than Control AA diet mice never exposed to a WD (FIG. 27). Insulin tolerance was similarly improved in both groups (FIG. 28), even as the mice continued to consume a high-calorie high-sugar diet. The improvements in glucose tolerance and insulin sensitivity of mice consuming diets with reduced levels of BCAAs were maintained over the course of the study (FIG. 29, 30). We observed no effect of extra dietary BCAAs (WD High BCAA) on either glucose tolerance or insulin sensitivity (FIG. 27-30).

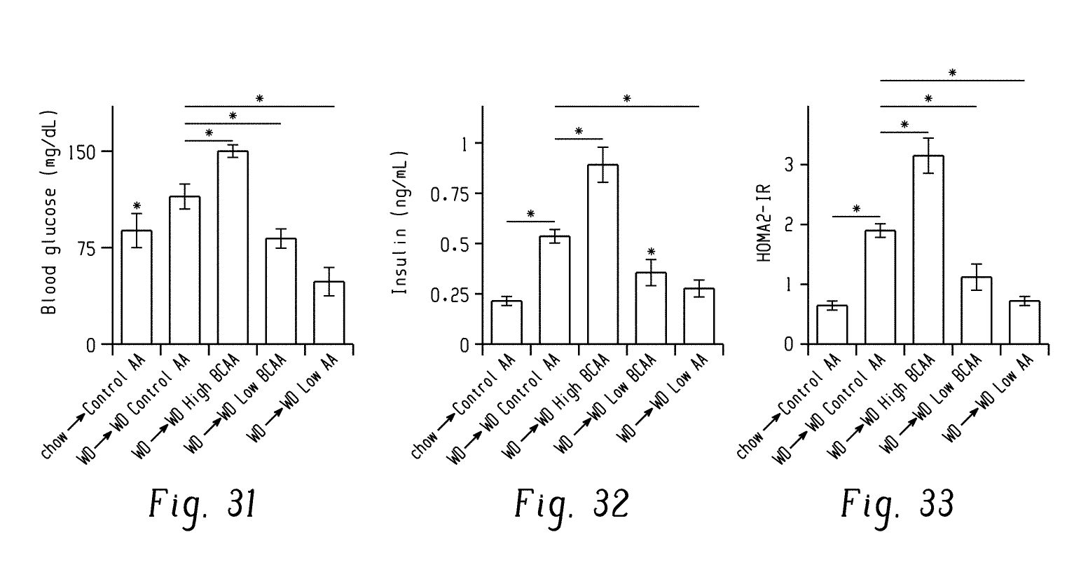

Mice on WD Low BCAA and WD Low AA diets had decreased fasting blood glucose and insulin levels (FIG. 31, 32). Conversely, increasing dietary BCAAs resulting in fasting hyperglycemia and hyperinsulinemia. HOMA2-IR calculated from these values indicate that insulin sensitivity is inversely correlated with dietary BCAA levels (FIG. 33).

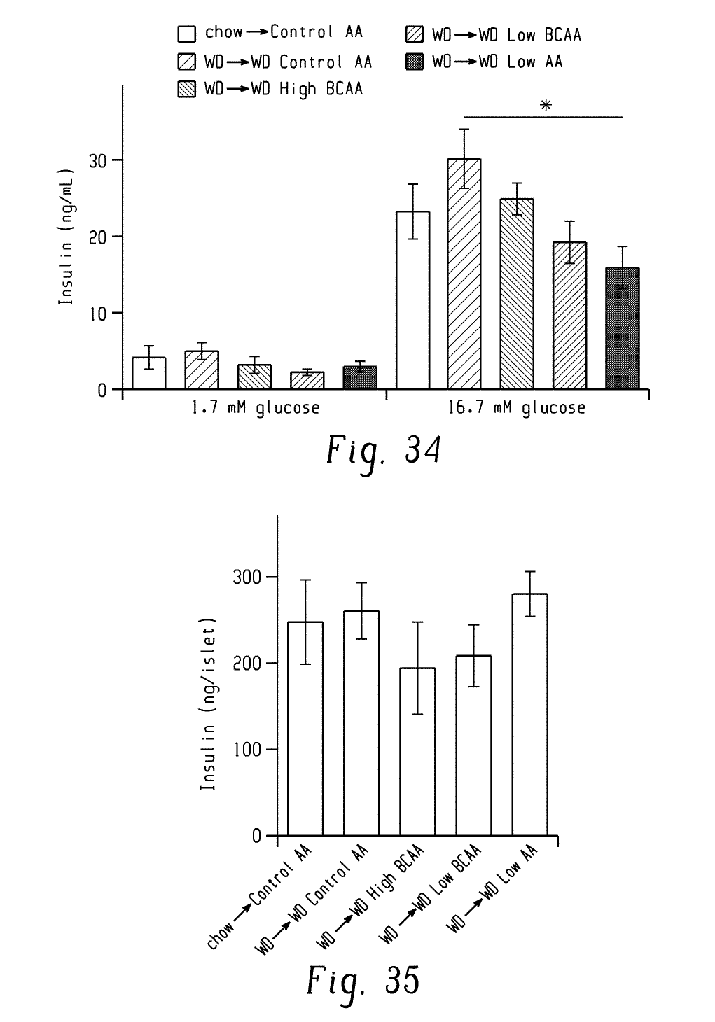

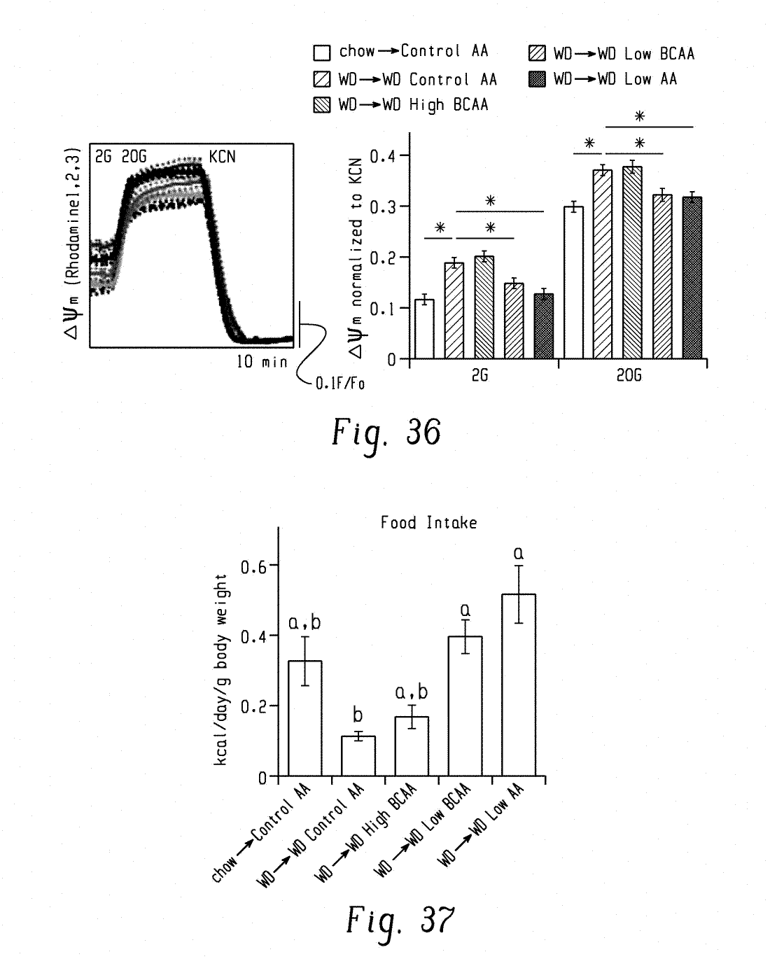

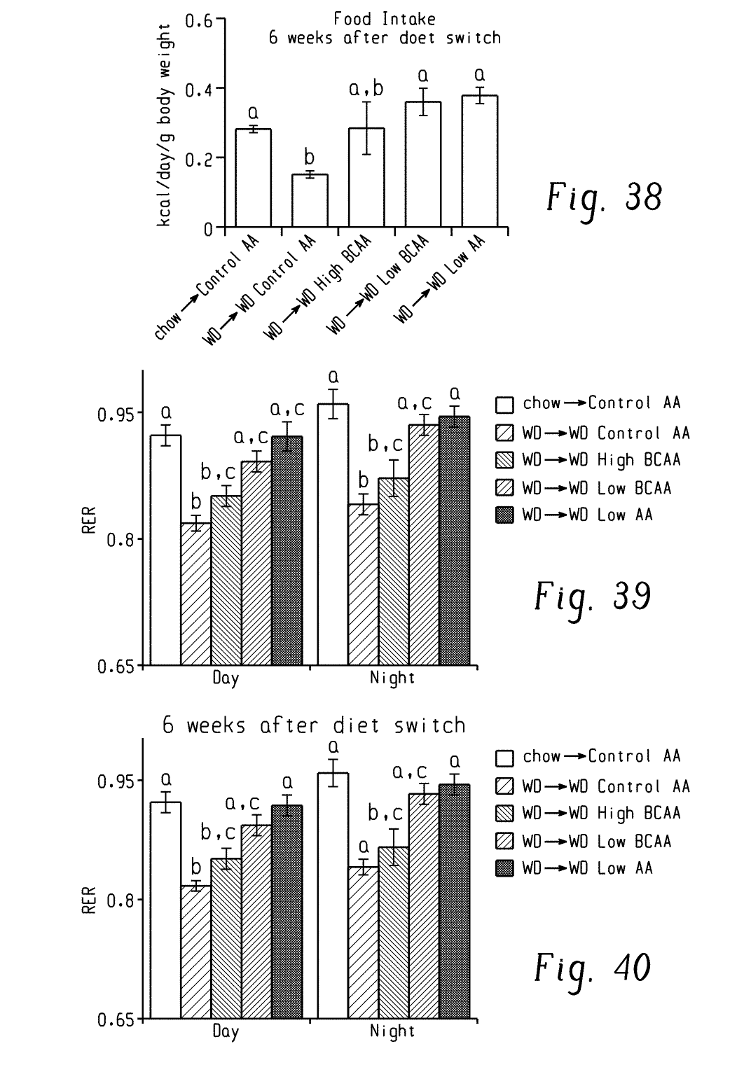

In order to examine the metabolic health of the pancreatic beta cells, pancreatic islets were isolated from mice in each diet group and an ex vivo glucose-stimulated insulin secretion assay was performed. In agreement with our previous studies in mice on a normal-calorie diet, we observed decreased insulin secretion in mice on a WD Low AA diet (FIG. 34); however, total islet insulin content was not affected (FIG. 35). For a more precise examination of beta cell metabolic stress, we quantified mitochondrial membrane potential (MMP) in both low and high glucose conditions. As expected, MMP was increased in mice eating a WD Control AA diet, and was reduced and essentially normalized in mice consuming WD Low BCAA and WD Low AA diets (FIG. 36). In every case except WD High BCAA, we noted that beta cell function was matched with insulin sensitivity, implying that the WD High BCAA diet may negatively impact mitochondrial function and insulin secretion.

### Example 3: Chronic Consumption of a Reduced BCAA Western Diet Increases Energy Expenditure Independently of FGF21

In order to understand the metabolic basis by which diets with reduced BCAAs promote leanness, metabolic chambers were utilized to examine food consumption, respiration, activity, and energy expenditure after mice had been on the diets for approximately 6 weeks and approximately 12 weeks. In line with our observation that weight loss in mice switched to diets with reduced levels of BCAAs was not due to decreased food consumption, mice fed WD Low BCAA and WD Low BCAA diets consumed about twice as many calories relative to their body weight than mice fed a WD Control AA diet (FIG. 37, 38). The respiratory exchange ratio (RER) was decreased for WD Control AA mice relative to mice never preconditioned with a Western diet, and the RER was increased in mice consuming the higher carbohydrate WD Low AA diet (FIG. 39, 40). Intriguingly, the RER of mice consuming the WD Low BCAA diet were indistinguishable from mice consuming a WD Low AA diet, despite the much higher level of calories derived from carbohydrates in the WD Low AA diet.

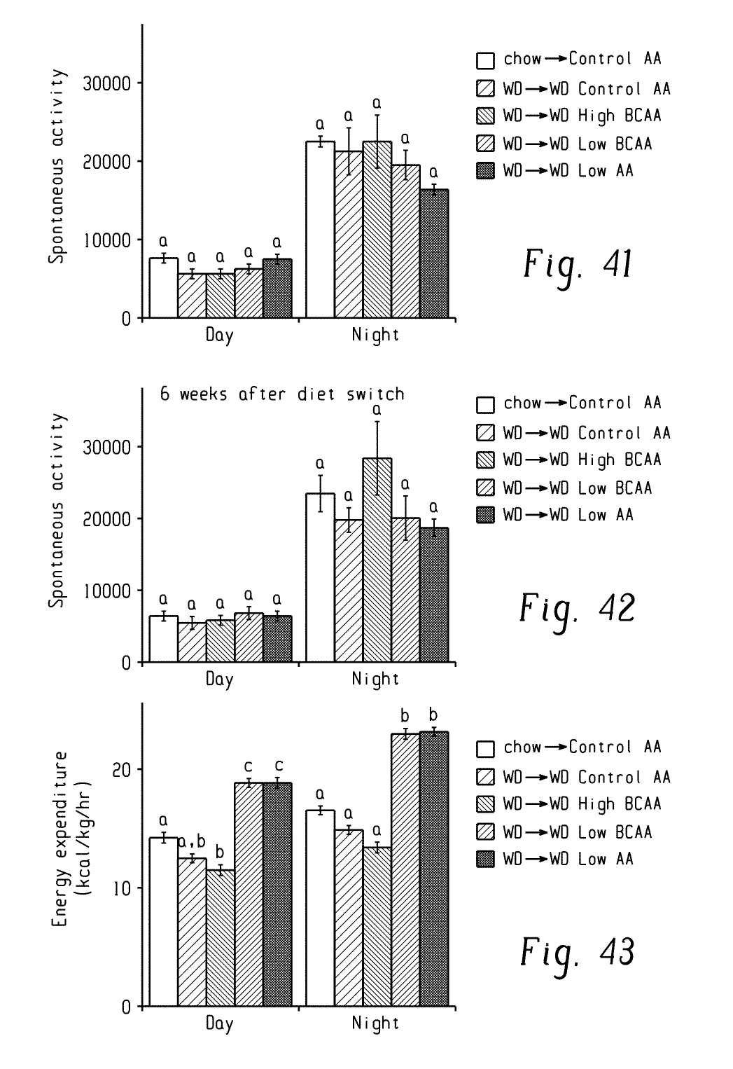

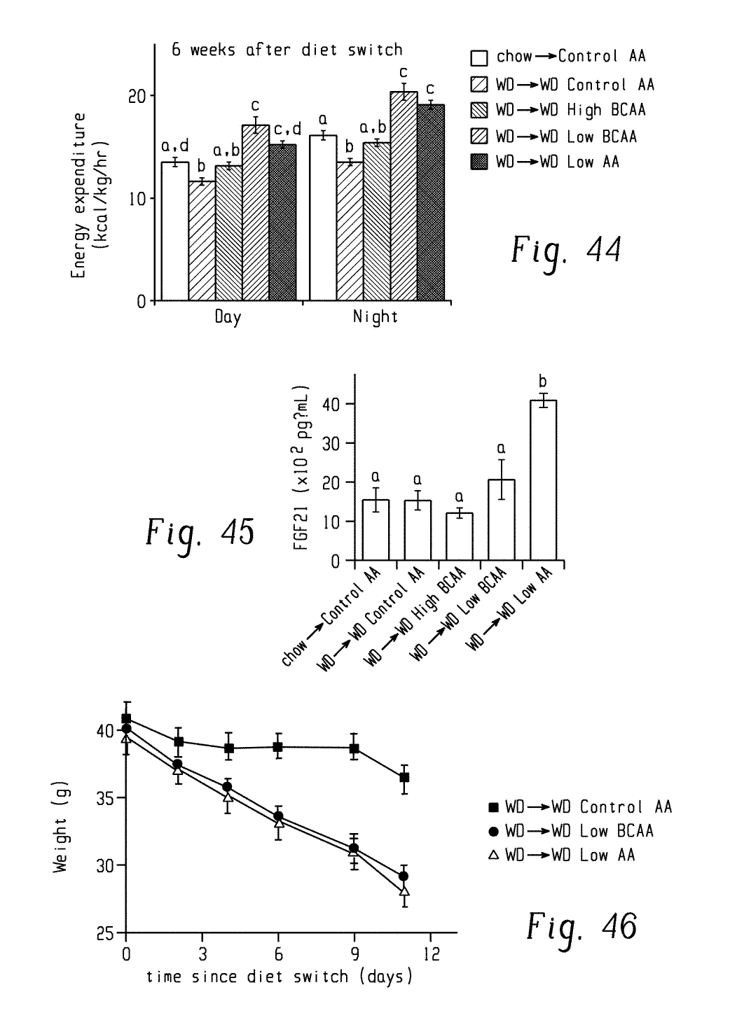

There was no difference in spontaneous activity between any of the groups of mice (FIG. 41, 42), but significant differences in energy expenditure were observed, with mice consuming the WD Low BCAA and WD Low AA diets having significantly greater energy expenditure during both the daytime and the nighttime (FIG. 43, 44). Increased energy expenditure following protein or total AA restriction is mediated by increased expression of the hormone FGF2; it was previously demonstrated that reducing dietary BCAAs in the context of a normal calorie diet did not increase energy expenditure or induce FGF21. Given the increased energy expenditure of mice on the WD Low BCAA diet, we determined the level of FGF21 in the blood of all groups. In agreement with previous results and the literature, mice fed a diet in which all amino acids were reduced (WD Low AA) had high levels of FGF21 (FIG. 45); however, there was no increase in FGF21 levels in mice consuming the WD Low BCAA diet.

### Example 4: Switching to Western Diets with Reduced Levels of the BCAAs is Accompanied by a Transient Increase in FGF21 Levels

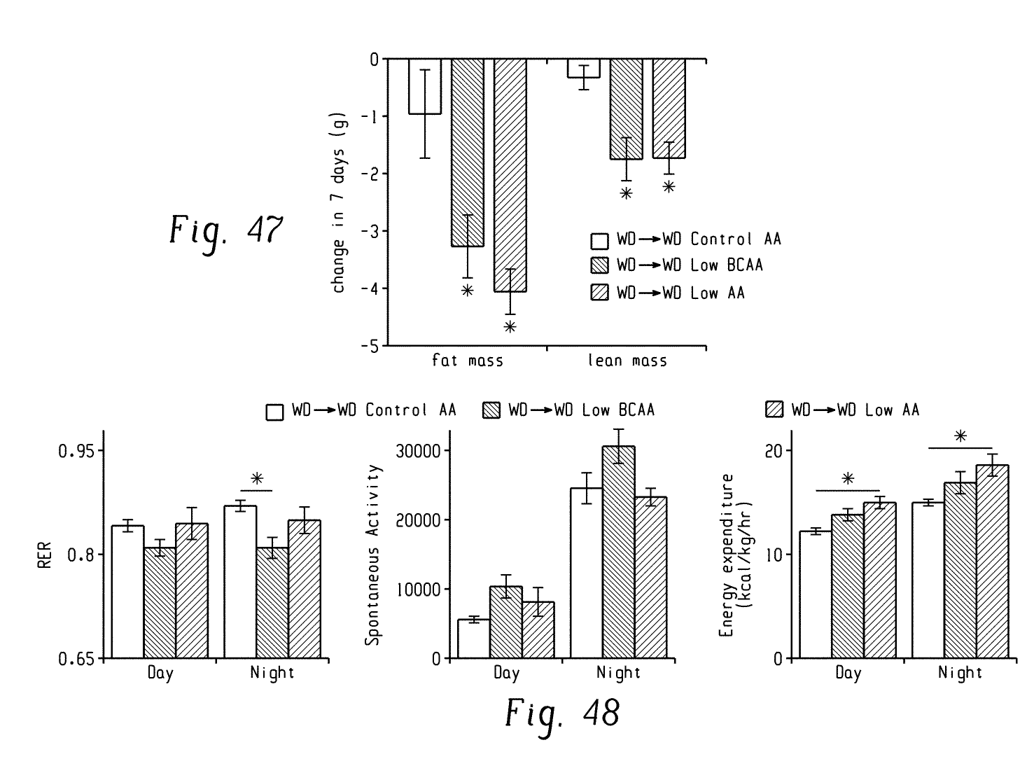

From the perspective of weight and body composition, the three weeks following the diet switch is distinctly different from the time period (six to twelve weeks after the diet switch) during which we conducted the analysis of food consumption and energy expenditure discussed above. In particular, rapid weight normalization occurs during the first three weeks, while weights are relatively stable thereafter. In order to analyze this time period of rapid weight loss, an additional cohort of mice were preconditioned and weight, body composition, activity and energy expenditure during the twelve days immediately following the diet switch were analyzed. As shown in FIG. 46, mice consuming the WD Low BCAA and WD Low AA diets progressively lost weight during this time period. As expected, the majority of this weight loss was due to a decrease in fat mass (FIG. 47).

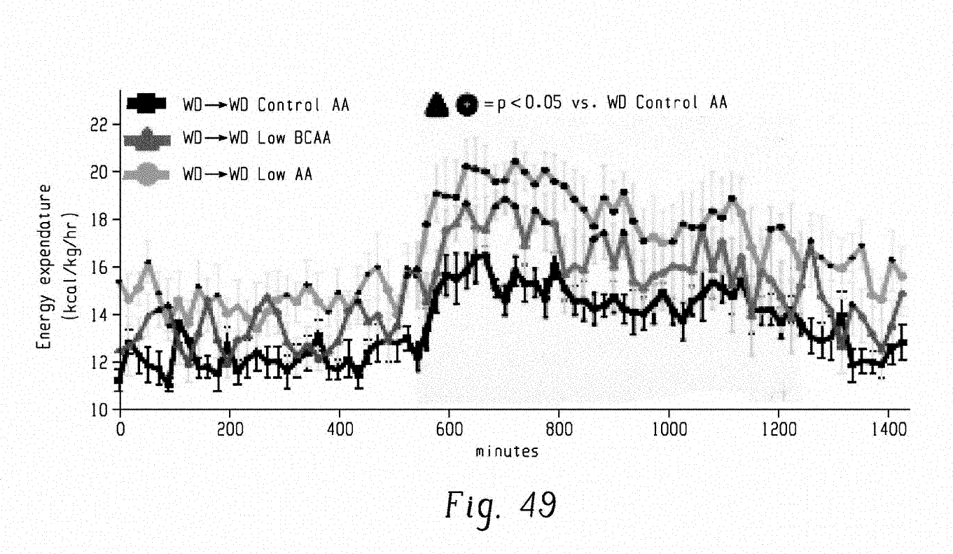

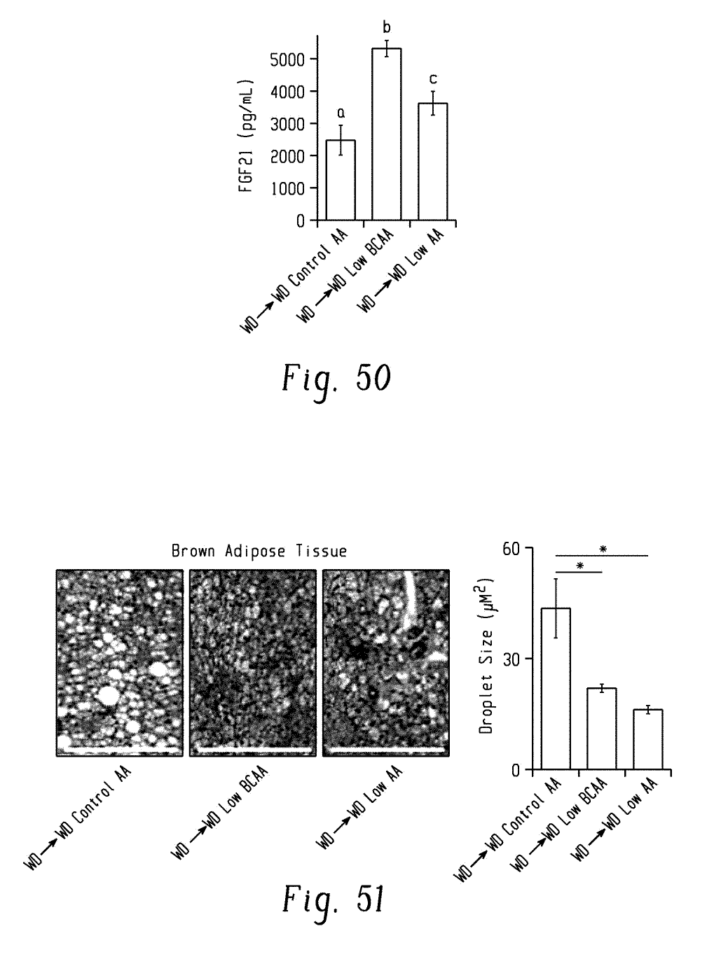

In contrast to the increased RER seen at later time points, neither WD Low BCAA or WD Low AA diet mice had increased RER one week after the diet switch; indeed, WD Low BCAA mice had lower RER than WD Control AA fed mice one week after the diet switch (FIG. 48). While there were no significant changes in spontaneous activity between groups, we observed a statistically significant increase in energy activity during daytime or nighttime in WD Low AA diet mice but surprisingly not in WD Low BCAA fed mice (FIG. 48). As weight loss in the absence of a change in energy expenditure was puzzling, we examined energy expenditure more closely throughout the course of a 24-hour period. Mice fed the WD Low BCAA diet do have a significant increase in energy expenditure for over 20% of a 24-hour period, with the most pronounced difference at night (FIG. 49). During this period of rapid weight loss, FGF21 was significantly increased in the blood of WD Low BCAA diet mice as well as in the blood of WD Low AA diet mice (FIG. 50). Intriguingly, FGF21 levels were highest in WD Low BCAA diet mice, while energy expenditure was highest in WD Low AA diet mice.

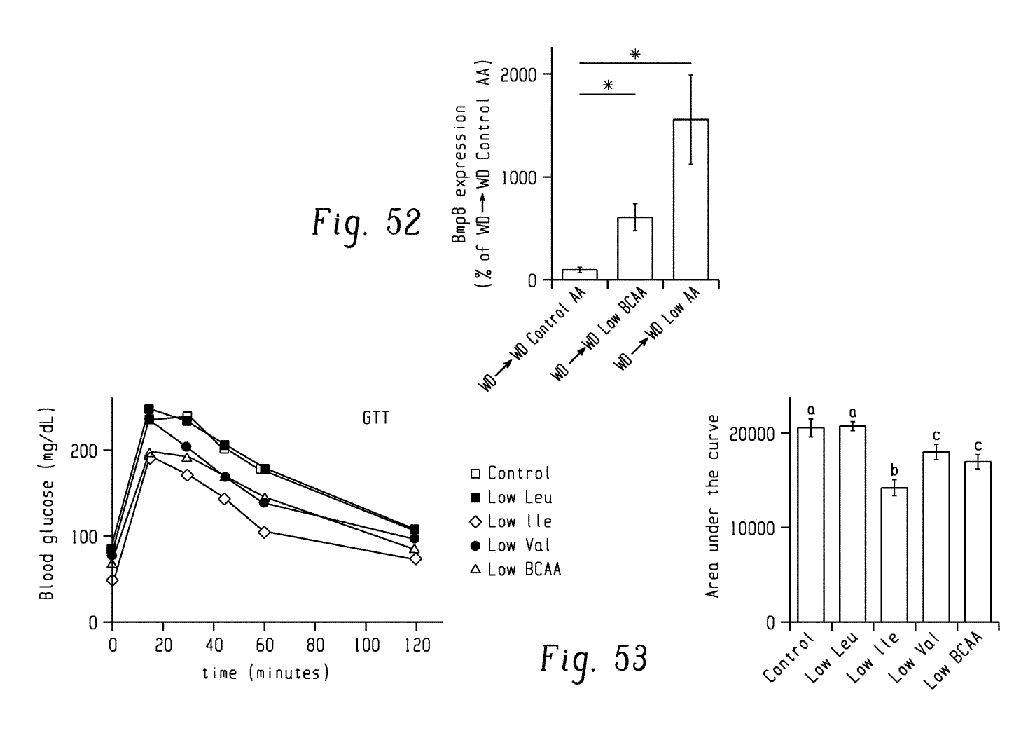

The increased energy expenditure mediated by FGF21 is associated with browning of white adipose tissue and increased activation of brown adipose tissue. A significant decrease in lipid droplet size (FIG. 51) and increased expression of Bmp8 (FIG. 52) were observed, changes consistent with FGF21-mediated activation of brown adipose tissue.

### Discussion of Examples 1-4

The experiments presented herein tested the hypothesis that reducing dietary BCAAs would be a uniquely potent way to intervene in metabolic syndrome. It was found that specifically reducing dietary BCAAs rapidly restores metabolic health, with obese mice normalizing their weight and fat mass within a month even while continuing to consume a high-fat, high-sugar Western diet. Mice fed diets with reduced levels of BCAAs also showed dramatic improvements in glucose tolerance, and insulin sensitivity, and with time also showed improvements in hepatic steatosis. Conversely, BCAA supplementation had no clear beneficial effects on metabolic health.

These findings represent the first time that specifically reducing dietary BCAAs has been tested as an intervention in the context of continued consumption of a high-calorie, high-fat, high-sugar “Western” diet. This approach allowed us to specifically address the consequences of reducing BCAAs without altering energy density or the caloric contribution of amino acids to the diet. We find that dietary reduction of BCAAs rapidly restores metabolic health even when mice continue to consume an energy dense Western diet; this suggests that a pharmacological intervention that mimics the effect of BCAA restriction, perhaps by altering BCAA uptake or catabolism, could be extremely efficacious in promoting metabolic health. In support of this hypothesis, mice with a global defect in BCAA catabolism have improved glucose tolerance and increased energy expenditure.

In addition, these findings represent the first time that specifically reducing dietary BCAAs has been tested as an intervention in the context of pre-existing obesity and glucose intolerance.

While we do not pinpoint a single reason for the metabolic benefits of a reduced BCAA diet, the improvements in glucose homeostasis we observe here may be due in part to weight loss and decreased adiposity, which in turn result from increased energy expenditure. While FGF21, a hormone that promotes energy expenditure, was strongly upregulated in mice fed WD Low BCAA and WD Low AA diets for approximately two weeks, increased levels of FGF21 were not observed in mice which consumed the WD Low BCAA diet for several months, despite extremely high increases in energy expenditure.

Without being held to theory, one particularly interesting effect that likely contributes to improvements in glucose tolerance and promotes hepatic insulin sensitivity is the effects of reducing dietary BCAAs on hepatic lipids. It is likely that the improved glucose tolerance following dietary BCAA reduction can be attributed at least in part to improved hepatic insulin sensitivity; defining the precise contribution of improved insulin sensitivity in the liver and altered glucose uptake into skeletal muscle and adipose tissue will require quantitatively assessing glucose production and disposal. Intriguingly, while dietary protein has been suggested to lower the risk of developing non-alcoholic fatty liver disease, and studies have suggested that BCAA supplementation may be therapeutic for hepatic steatosis, we find that specifically reducing dietary BCAAs reduces hepatic lipid content and smaller lipid droplets. Notably, reducing all the dietary amino acids—as in a low protein diet—did not reduce hepatic lipid droplet size, demonstrating that specifically reducing BCAAs may have uniquely beneficial effects on hepatic lipid metabolism.

Here, we have demonstrated for the first time that specifically reducing dietary levels of the three branched-chain amino acids, leucine, isoleucine, and valine can rapidly reverse the obesity and metabolic dysfunction resulting from consumption of a high-fat, high-sugar diet. Pharmaceuticals which mimic the effects of a reduced BCAA diet, or selective reduction of dietary BCAAs through the use of BCAA-free medical foods are approaches to promote metabolic health and treat diabetes and obesity than reducing caloric intake.

### Example 5: Reducing Dietary Isoleucine or Valine

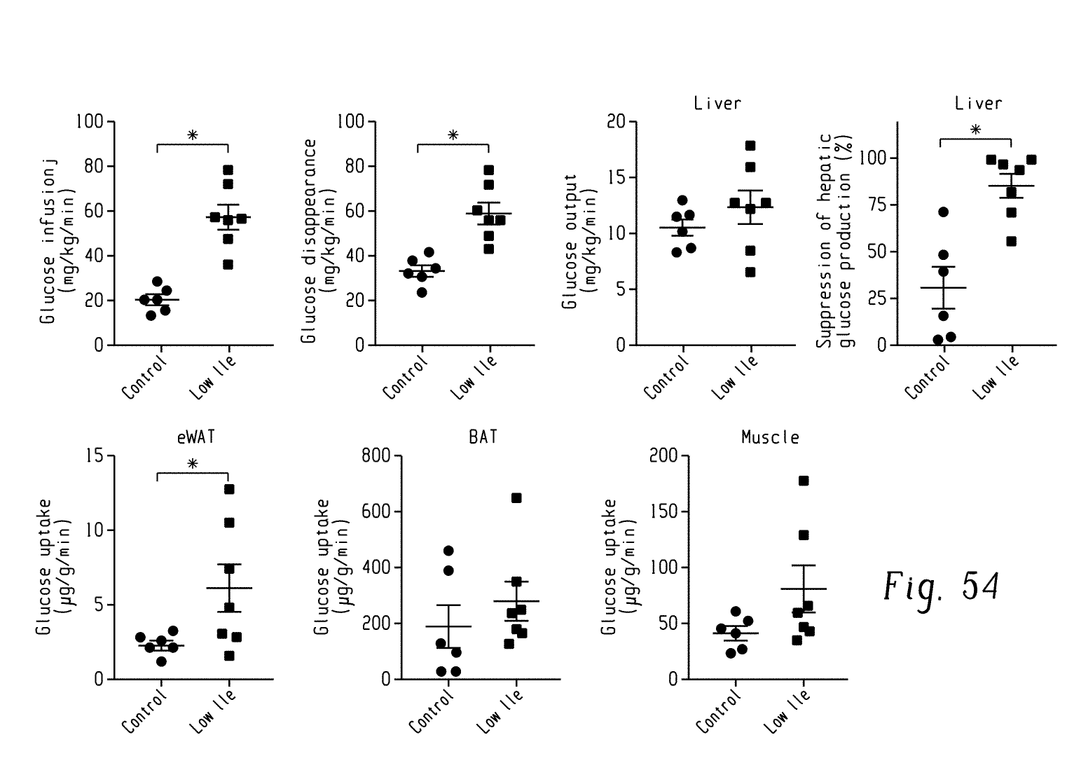

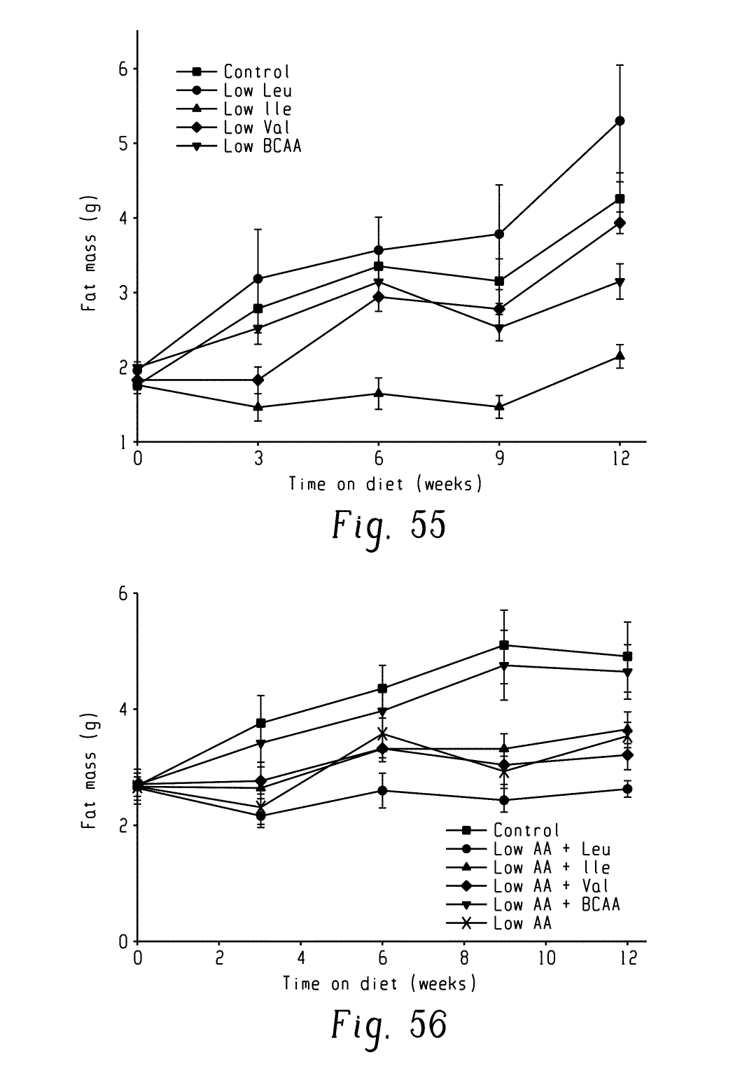

The effect of specifically reducing levels of each individual branched chain amino acid was determined. Specific reduction of either isoleucine or valine by 67% is sufficient to improve glucose tolerance (FIG. 53), with reduction of dietary isoleucine being particularly potent. To examine the specific effect of dietary isoleucine on hepatic insulin sensitivity, we euglycemic-hyperinsulinemic clamps were conducted in collaboration with Dr. Joseph Baur's clamp facility at U. Penn. Reducing dietary isoleucine improved hepatic insulin sensitivity while also stimulated glucose uptake into white adipose tissue (FIG. 54). Male mice consuming a low isoleucine diet also showed decreased fat mass gain compared to both Control mice and mice on a Low BCAA diet (FIG. 55).

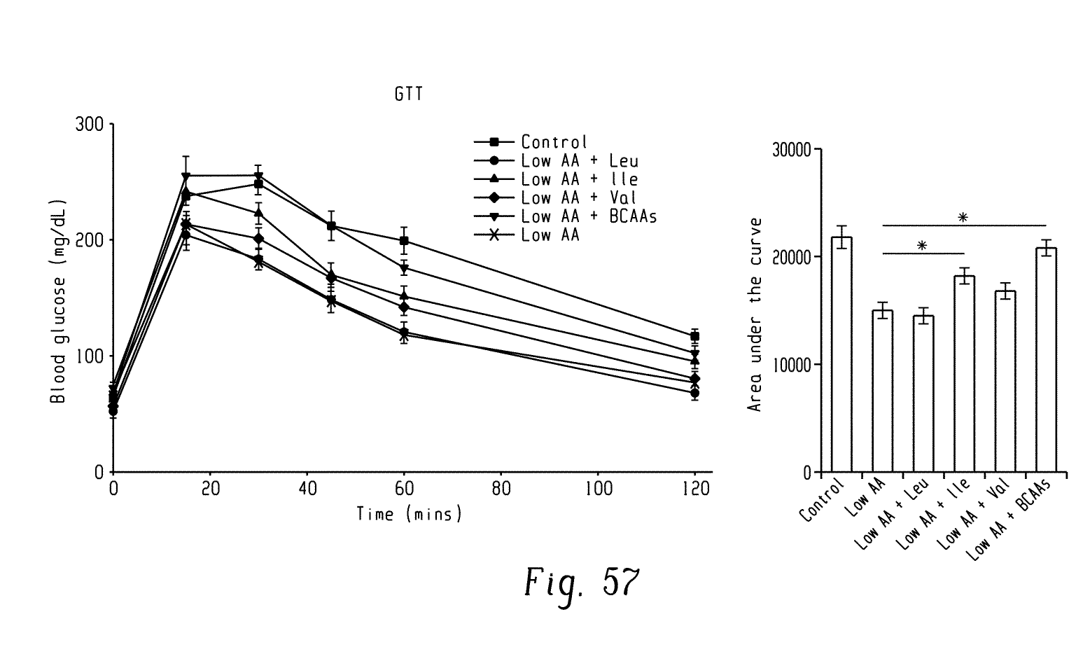

The reverse experiment to determine if reduction of dietary isoleucine is essential for the metabolic benefits of a low protein diet was also performed. We determined that “adding back” normal levels of the BCAAs to a low protein diet blocked the effects of a Low AA diet on fat mass gain by lean mice (FIG. 56). Intriguingly, leucine supplementation of a Low AA diet seemed to potentiate the effect of a Low AA diet, blocking fat mass gain even more effectively. Adding back normal levels of dietary Isoleucine, but not leucine or valine, likewise blocks the effects of a Low AA diet on glucose tolerance (FIG. 57).

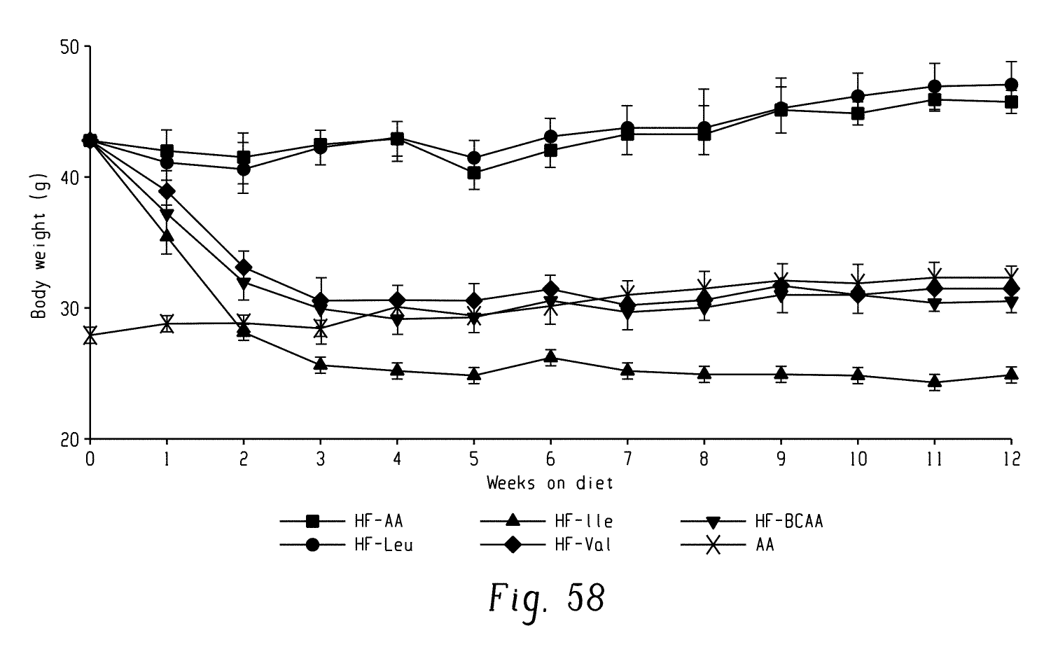

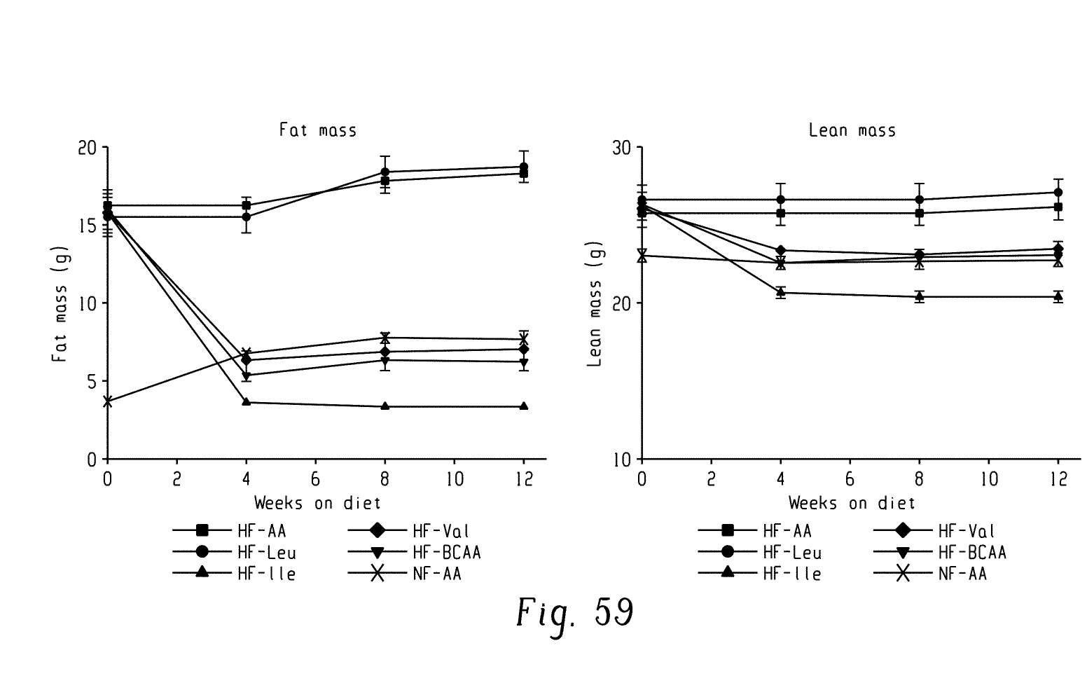

These data highlighted a critical role of dietary isoleucine, and to a lesser extent valine, in the metabolic response to branched-chain amino acids. We next decided to test the effects of the individual BCAAs on the metabolic response of diet-induced obese mice. As shown in FIG. 58, specifically restricting either isoleucine or valine recapitulated the effects of restricting all three BCAAs; indeed, restriction of isoleucine led to more rapid weight loss than restriction of all BCAAs, although restriction of isoleucine resulted in some lean mass loss, while restriction of either valine or all BCAAs did not (FIG. 59).

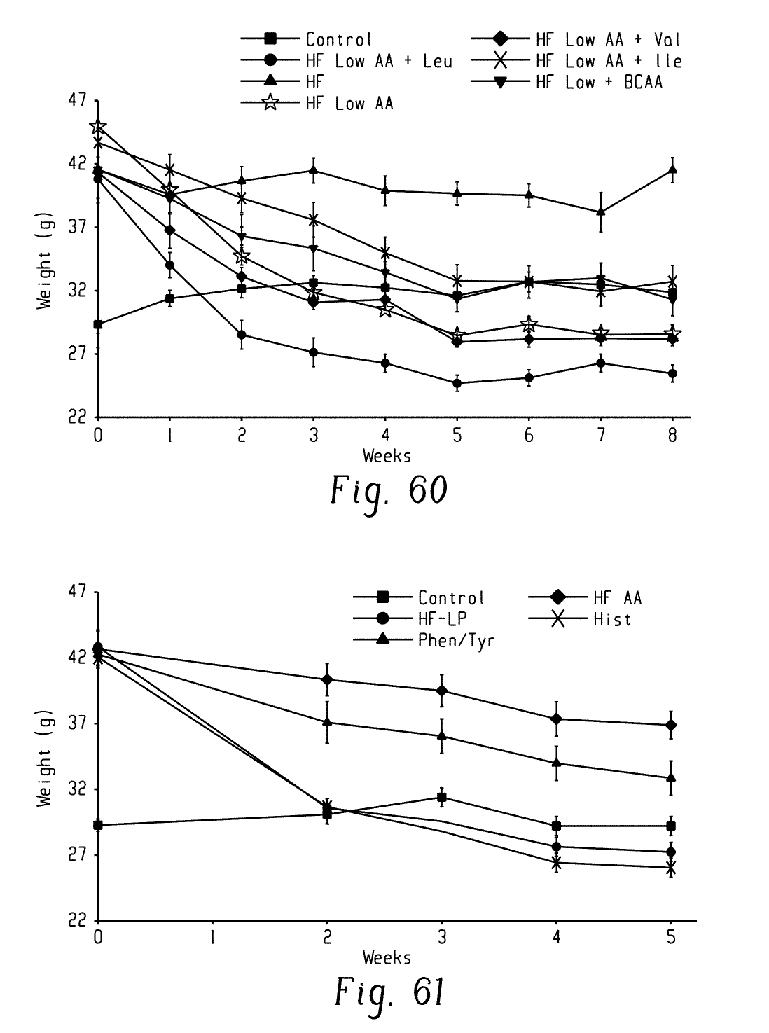

We also tested the role of the individual BCAAs in diet-induced obese mice by “adding back” individual BCAAs to obese mice fed a low amino acid, high-fat high-sucrose diet. As shown in FIG. 60, adding back isoleucine or all three BCAAs to a low AA diet slows the weight loss of mice fed a low amino acid high fat, high-sucrose diet. However, addition of leucine to a low AA, high fat high sucrose diet led to extremely rapid weight loss—such that the mice reached a normal body weight twice as rapidly as when all amino acid were restricted.

### Example 6: Effects of Essential Amino Acids on Body Composition and Glycemic Control

To further explore the effect of the individual essential amino acids on body composition and glycemic control, we examined the effect of reducing each of the six other essential dietary amino acids by ⅔rds in lean mice. We did not observe improved glycemic control following restriction of any other single essential amino acid; however, we noticed that mice in which we restricted histidine (His) or we restricted both phenylalanine and tyrosine (Phe/Tyr) the mice had reduced weight gain. We examined if restriction of either histidine or of Phe/Tyr could cause weight loss or restoration of metabolic health. As shown in FIG. 61, specifically restricting either His or Phe/Tyr resulted in weight loss in diet-induced obese mice, with His restriction resulting in rapid weight loss and Phe/Tyr a slower, more gradual reduction in weight.

### Example 7: Human Study of the Benefits of Low BCAA Diet

Subjects will be male between the ages of 35-65, with a BMI of 28-35 (mildly obese/overweight), with a fasting glucose level of 101-125 mg/dL, a stable weight of within 5 pounds for at least 3 months, not taking or willing to cease taking over the counter supplements, and not planning to begin a diet or exercise program.

Subjects will be randomized to one of two groups, low BCAA diet vs. control diet. The low BCAA diet group will receive BCAD2 (Mead Johnson), a fortified medical food powder that does not contain the branched chain amino acids isoleucine, leucine, or valine. BCAD2 is routinely used for the treatment of adults with MSUD, and provides all other essential amino acids, as well as nonessential amino acids, carbohydrates, fat, vitamins, and minerals including iron. The control diet group will be provided with a protein supplement in powder form (Abbott Labs) which will provide all amino acids. Diets will be provided in unmarked containers, to ensure subjects will be blinded to the dietary group assignment.

Recipes have been developed for the subjects to use to mix the powders with other liquids to generate a meal replacement beverage. This food preparation strategy will maximize palatability and match energy density and total protein content between the control and the low BCAA diets. The subjects will be able to use multiple different recipes over the course of the study to prevent taste fatigue and dropout. We anticipate that each subject will replace 2 meals per day with a beverage. An individual consuming 2600 calories/day would therefore consume approximately 1700 calories/day, and approximately ⅔rds of their daily protein, in the form of meal-replacement beverage. Individualized dietary instructions will be generated on the basis of a four-day food diary subjects will complete prior to the enrollment in the study. Subjects may be counseled regarding suggested protein intake for the remaining meal of the day, with the goal of maintaining a similar level of total daily protein intake, and will complete a four-day food diary each month to assess total caloric intake and macronutrient composition. Subjects will return diet containers monthly and the remaining product will be measured to assess compliance.

Initial Telephone Contact: Subjects will be asked to self-report height and weight. Directed questions will be asked to determine if potential subjects meet the inclusion or exclusion criteria of the study. Specifically, subjects will be asked about a diagnosis of diabetes or the use of any medications for diabetes or weight loss. We will ask subjects if they have had screening for diabetes performed with a fasting glucose value or hemoglobin A1C. Subjects will be asked to self-report the results of this testing if it was done in the past year and they are aware of the results. The study design will be explained, including overall schedule of visits and procedures. If potential subjects would like to proceed with screening to determine eligibility for the study, they will provide oral consent for completion of food diary, overnight fasting, and medical records review and will be scheduled for Study Visit 1, where clinical labs and a palatability test will be performed to determine eligibility for the study. A blank four-day food diary will be mailed to their home with instructions to document their usual food intake (over consecutive days if possible; one day should be a weekend day) and subjects will complete this food diary prior to Study Visit 1.

Study Visit 1:

Subjects will be asked to arrive in the morning after an overnight fast of at least 8 hours. Height and weight will be measured. Body mass index (BMI) will be calculated. If BMI is not within inclusion range, subjects will be excluded from the study before further procedures are performed. If a fasting glucose value was not available within the past 6 months on medical record review, we will check fasting glucose with glucometer prior to further study procedures. If fasting glucose is less than 95 mg/dL and the subject does not have clear history of elevated fasting glucose, elevated hemoglobin A1C or impaired glucose tolerance they will be excluded from the study. If fasting glucose is >126 mg/dL, we will send fasting clinical labs for only glucose and hemoglobin A1C. The subject will not have any further study procedures until we can confirm whether they meet criteria for a diagnosis of diabetes (glucose ≥126 mg/dL or Hemoglobin A1C ≥6.5%) (13). If so, a team member will notify the subject and they will be excluded from the study. Subjects will have a review of all their current medications, medical history, and recent weight changes with study team members. No more than 30 mL of blood will be collected from a peripheral vein to measure glucose, hemoglobin A1C, complete blood count, albumin, pre-albumin, total protein, ALT and AST. Before samples are sent to the clinical lab for processing, subjects will be provided with samples of the meal replacement beverages for both the low BCAA and the control diets. Subjects will taste these samples and then be asked to indicate their willingness to continue with the study based on palatability. This will avoid early subject dropout due to personal taste issues. Food diary review will ensure subjects meet minimum required protein intake. Subjects will also receive brief instructions for completion and brief instruction on meal replacement beverage preparation/recipes to ensure these will be feasible for the subjects in their usual home/work environment. Height and weight will be measured. Following Visit 1, clinical labs and patient history will be reviewed by MD team member. If patients meet the inclusion criteria and no exclusion criteria are identified as a result of patient history, clinical labs, or food diary review, and the patients find the beverage samples palatable, the subjects will be invited to begin the study and return for Study Visit 2. If excluded for other reasons, patients will be informed as to the reason for their exclusion, and told to follow up with their primary care providers for further workup and treatment if appropriate.

Study Visit 2:

Subjects will return in the morning after an overnight fast of at least 8 hours. Height, weight and waist circumference will be recorded. An IV will be placed in a peripheral vein to allow for access for repeated blood draws during the glucose tolerance test. Up to 30 mL of a fasting blood sample will be collected.

Next, resting metabolic rate (RMR) will be measured with a Deltatrak Respiratory Gas Analyzer, calibrated before each measurement using known gases of 95% oxygen and 5% CO₂. This portable unit measures the concentration of oxygen and carbon dioxide in air streams entering and exiting a clear plastic hood (canopy) placed over the subject's head. The fasting subject will lie quietly for 30 minutes before and then remain in bed for the entire duration of this procedure. Following a 10-minute period of adjustment to the monitoring conditions, respiratory gas exchange will be measured and averaged over 30 minutes. Despite the clear plastic hood, a very small percentage of subjects feel claustrophobic. As such, we exclude participants who self-report being claustrophobic. Should a claustrophobic participant somehow avoid being screened out, such participants can easily push the light-weight hood away from their face.

Next, an oral glucose tolerance test will be performed. The subject will consume 75 grams of glucose orally within 10 minutes. Blood draws will be collected for glucose and insulin measurements at 30, 60, and 120 minutes after the start of glucose ingestion. These samples will be sent to the UW clinical laboratory. If any subjects have a 120 minute glucose value ≥200 mg/dL (suggestive of a diagnosis of diabetes (13)) the subjects will be informed of this result by MD study team members and advised to discuss with their primary care providers. The subjects will continue in the study however, unless their fasting glucose and/or hemoglobin A1C values also confirm a diagnosis of diabetes or they are started on a medication to treat diabetes by another provider. A 2 hour glucose value ≥200 mg/dL is not independently diagnostic for diabetes, and must be confirmed by repeat and/or additional testing.

After these procedures are completed, the subjects will be allowed to eat a meal. Dual energy X-ray absorptiometry (DXA), a standard technique for the determination of body composition, including fat mass, will be performed.

At the end of this visit, the subjects will be randomized and given the appropriate amount of powdered supplement for a 1-month supply, pre-weighed in multiple containers, or original product containers with labels removed and new labels attached.

Subjects will begin the study as soon after visit 2 as feasible (ideally the following day).

2-3 days after Visit #2: Subjects will be contacted and asked about compliance with the diet, any concerns about diet preparation, and any dietary questions. Subjects will be asked about any side effects, including muscle weakness, easy bruising, light-headedness, etc. The subjects will be encouraged to continue good compliance with the diet.

Approximately 10 days before Visit #3, subjects will be asked to complete another 4-day food diary.

The day prior to Visit #4, subjects will be asked to collect a stool sample using a provided kit and bring with them to their visit.

Visit #3 (after ˜30 days on diet): We will review food diary, review food preparation instructions, modify amount of food replacement daily if needed or suggest modifications to other food intake during the day. We will have subjects complete a survey about the acceptability of the meal replacement beverages. Fasting blood draw of up to 30 mL will be performed for research sample (to be banked for now), glucose, total protein, albumin, prealbumin, ALT, AST. These results will be reviewed by MD on study team. If total protein, albumin, or prealbumin has fallen below the normal range, subjects will be asked to incorporate more protein in their regular dietary food intake. Height, weight, and waist circumference will be measured and recorded. We will again do a review of any medications (prescribed or over the counter), symptoms, other illnesses. The food containers dispensed at Visit #2 will be returned and weighed to assess compliance. We will provide another 1-month supply of meal replacement product. Approximately 10 days before Visit #4, subjects will be asked to complete another 4 day food diary. The day prior to Visit #4, subjects will be asked to collect a stool sample using a provided kit and bring with them to their visit.

Visit #4, repeat of Visit#2 (after ˜60 days on diet): The procedures conducted during visit 2 will be repeated, including fasting blood draw for clinical and research analysis, height, weight and waist circumference measurements, glucose tolerance test, RMR, DXA, BIS, jumping test and meal replacement beverage acceptability survey.

Visit #5—˜14-21 days after study completion: Subjects will return to CRU after overnight fast for clinical labs including fasting glucose, total protein, albumin, prealbumin, ALT, AST and collection of blood sample for research analysis (amino acid levels, other hormones, as above). Height, weight and waist circumference will also be recorded. The subjects will again be asked about any symptoms of adverse events, as asked during the study. Subjects will be instructed not to start any new diabetes or weight loss medications or over the counter supplements, or initiate any new diet and exercise programs until completion of study at visit #5. The purpose of this visit is to determine if the effects of diet modification are lost immediately after resuming a normal diet, or have some sustained effect.

Data will be collected by the study coordinator working with the subject to get the information and/or collecting this information from the medical record. Data will be stored in a secure fashion.

Risks of the BCAD2 medical food may include: dizziness, headache, fatigue, hypoglycemia (low blood sugar), skin rash, minimum loss of muscle mass (due to weight loss), muscle weakness, neuropathy, or mood changes. These are all classified as unlikely risks. Total protein and albumin levels will be monitored at the 30 day timepoint. If total protein or albumin has fallen below the normal range, the subject will be contacted and overall protein intake will be reviewed. We will recommend increased protein intake through their regular diet. These risks are considered very unlikely.

It is expected that the meal replacement beverages will be a feasible method of reducing dietary BCAA consumption. In addition, it is expected that subjects will exhibit one or more of the following: reduced weight, reduced fat mass, and/or reduced fasting blood glucose levels, while retaining lean mass and muscle strength.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of improving metabolic health in a mammalian subject in need of improvement in metabolic health, comprising
reducing the mammalian subject's consumption of branched chain amino acids (BCAAs) by the mammalian subject consuming a reduced BCAA daily diet, wherein the reduced BCAA daily diet has the following characteristics:
a percentage of calories from BCAAs consumed during the course of the day are reduced compared to a percentage of calories from BCAAs in a standard daily diet for the mammalian subject,
the daily intake of BCAAs in the reduced BCAA daily diet provides at least the recommended daily allowance of isoleucine, leucine and valine for the mammalian subject
the reduced BCAA daily diet includes all essential amino acids, and optionally non-essential amino acids,
the percentage of calories from non-BCAAs in the reduced BCAA daily diet are not substantially reduced compared to the percentage of calories from non-BCAAs in the standard daily diet for the mammalian subject, and
the reduced BCAA daily diet has at least the same number of calories as the standard daily diet for the mammalian subject, and
wherein the individual is not suffering from maple syrup urine disease.

2. The method of claim 1, wherein the reduced BCAA daily diet is reduced in leucine, isoleucine and valine; the reduced BCAA daily diet is reduced in both isoleucine and valine; the reduced BCAA daily diet is reduced in isoleucine only; or the reduced BCAA diet is reduced in valine only.

3. The method of claim 1, wherein the mammalian subject is a human subject who is overweight, obese, insulin-resistant, has elevated blood glucose levels, pre-diabetes, diabetes, has impaired glucose tolerance, has cardiovascular disease, is suffering from fatty liver disease, or a combination thereof.

4. The method of claim 1, further comprising determining BCAA content in the standard daily diet for the mammalian subject and reducing the BCAA content in the standard daily diet to provide the reduced BCAA daily diet.

5. The method of claim 4, wherein the standard daily diet is a reference diet based on the average caloric intake and average protein consumption for the mammalian subject, adjusted for weight, and optionally age, and the method further comprises determining BCAA content in the reference daily diet and reducing the amino acid content in the standard daily diet to provide the reduced BCAA diet.

6. The method of claim 1, wherein the wherein the percentage of calories from BCAAs in the reduced BCAA daily diet are reduced by at least 25% compared to the percentage of calories from BCAAs in the standard daily diet for the mammalian subject

7. The method of claim 1, wherein the mammalian subject is a human and the recommended daily allowance is 19 mg/kg/day of isoleucine, 42 mg/kg/day of leucine and 4 mg/kg/day of valine

8. The method of claim 1, wherein consuming is continued for a period of time sufficient to result in weight loss in the mammalian subject, or wherein consuming is continued for a period of time sufficient to reduce fat while not substantially decreasing lean muscle mass in the mammalian subject.

9. The method of claim 1, wherein the mammalian subject is a human subject consuming a standard Western diet, a human subject consuming excess daily calories, or both.

10. The method of claim 9, wherein the reduced BCAA daily diet comprises one or more medical meal replacements per day, wherein the percentage of BCAAs in the medical meal replacements is less than 17.2 wt % of the mass of protein plus amino acids.

11. The method of claim 10, wherein the medical meal replacement is a medical meal replacement beverage, or a solid food product.

12. The method of claim 1, wherein the reduced BCAA daily diet comprises a low protein daily diet and a supplement comprising essential non-BCAAs, wherein the low protein daily diet has less than 9% of calories from protein.

13. The method of claim 12, wherein the supplement further comprises non-essential non-branched BCAAs.

14. The method of claim 12, wherein the low protein daily diet is prescribed by a medical professional.

15. The method of claim 12, wherein the supplement is in the form of a pharmaceutical composition, a beverage, or a food item.

16. The method of claim 1, wherein the mammalian subject is a domesticated animal selected from cat, dog, ferret, gerbil, guinea pig, hamster, minipig, mouse, pig, rabbit, rat, or horse.

17. The method of claim 16, wherein the domesticated animal is overweight, obese, has pre-diabetes, has diabetes, or a combination comprising one or more of the foregoing.

18. The method of claim 1, wherein the mammalian subject is an animal housed in a facility for research or public viewing such as an animal research facility, animal sanctuary, or zoo, and wherein the animal is overweight, obese, has pre-diabetes, has diabetes, or a combination comprising one or more of the foregoing

19. A method of improving metabolic health in a mammalian subject in need of improvement in metabolic health due to excess weight or poor control of blood glucose levels, comprising
administering to the mammalian subject a compound that inhibits at least a portion of the gastrointestinal absorption of one or more branched chain amino acids.

20. The method of claim 19, wherein the compound is an inhibitor of an L-type amino acid transporter.

21. The method of claim 20, wherein the compound is 2-aminobicyclo-(2,2,1)-heptane-2-carboxylic acid; (S)-2-amino-3-(4-((5-amino-2-phenylbenzo[d]oxazol-7-yl)methoxy)-3,5-dichlorophenyl) propanoic acid; ESK242; ESK246, or a combination thereof.

22. The method of claim 19, wherein the human subject is overweight, obese, insulin-resistant, pre-diabetic, diabetic, suffering from fatty liver disease, or a combination thereof.

23. A method of feeding a mammalian subject in need of weight reduction, comprising
reducing the mammalian subject's consumption of BCAAs by the mammalian subject consuming a reduced BCAA meal replacement at least once per day,
wherein the percentage of calories from BCAAs in the reduced BCAA meal replacement are reduced compared to the percentage of calories from BCAAs in a standard meal for the mammalian subject, and
wherein the reduced BCAA meal replacement includes the essential amino acids histidine, lysine, methionine, phenylalanine, threonine, and tryptophan.

24. The method of claim 23, wherein the reduced BCAA meal replacement comprises no BCAAs.

25. A method of feeding a mammalian subject in need of weight reduction comprises
the mammalian subject consuming a low protein daily diet supplemented with leucine, wherein the total amount of leucine in the low protein diet supplemented with leucine is greater than or equal to 1.15 wt % of the total calories and/or is greater than 6 grams of leucine per day,
wherein the low protein diet comprises less than 9% of total calories from protein, and
wherein the normal diet comprises greater than 15% of total calories from protein.

26. A method of improving metabolic health in a mammalian subject in need of improvement in metabolic health, comprising
reducing the mammalian subject's consumption either histidine alone or of both phenylalanine and tyrosine by the mammalian subject consuming a reduced amino acid daily diet, wherein the reduced amino acid daily diet has the following characteristics:
a percentage of calories from either histidine alone or of both phenylalanine and tyrosine consumed during the course of the day are reduced compared to a percentage of calories from either histidine alone or of both phenylalanine and tyrosine in a standard daily diet for the mammalian subject,
the daily intake of either histidine alone or of both phenylalanine and tyrosine in the reduced amino acid daily diet provides at least the recommended daily allowance of histidine, phenylalanine and tyrosine for the mammalian subject
the reduced amino acid daily diet includes all essential amino acids, and optionally non-essential amino acids,
the percentage of calories from amino acids other than histidine or phenylalanine and tyrosine in the reduced BCAA daily diet are not substantially reduced compared to their percentages in the standard daily diet for the mammalian subject, and
the reduced amino acid daily diet has at least the same number of calories as the standard daily diet for the mammalian subject.

27. A method of feeding a mammalian subject in need of weight reduction, comprising
reducing the mammalian subject's consumption of either histidine alone or of both phenylalanine and tyrosine by the mammalian subject consuming a reduced amino acid meal replacement at least once per day,
wherein the percentage of calories from histidine alone or of both phenylalanine and tyrosine in the reduced amino acid meal replacement are reduced compared to the percentage of calories from histidine alone or of both phenylalanine and tyrosine in a standard meal for the mammalian subject, and
wherein the reduced amino acid meal replacement includes all essential amino acids.

28. The method of claim 27, wherein the reduced amino acid meal replacement comprises glycomacropeptide.

29. The method of claim 27, wherein the reduced amino acid meal replacement contains natural proteins with reduced levels of specific amino acids but does not contain glycomacropeptide.


SEQUENCE LISTING (24 sequences)

SEQ ID NO: 1 - DNA, 18 bp, artificial
  primer
aaggctatgtgaaggatg

SEQ ID NO: 2 - DNA, 17 bp, artificial
  primer
gccagcgggagcacatc

SEQ ID NO: 3 - DNA, 20 bp, artificial
  primer
accttctacaatgagctgcg

SEQ ID NO: 4 - DNA, 21 bp, artificial
  primer
tcaacacaaccctccacatca

SEQ ID NO: 5 - DNA, 21 bp, artificial
  primer
tggtgtgtggtgatgctgatc

SEQ ID NO: 6 - DNA, 23 bp, artificial
  primer
agtggcaatgctatcatcatcat

SEQ ID NO: 7 - DNA, 20 bp, artificial
  primer
cccctctgttaattggctcc

SEQ ID NO: 8 - DNA, 20 bp, artificial
  primer
caaatcctgggtgtcaaagc

SEQ ID NO: 9 - DNA, 19 bp, artificial
  primer
caacaccatccccgacatc

SEQ ID NO: 10 - DNA, 21 bp, artificial
  primer
gtactgccgttttcacaagtg

SEQ ID NO: 11 - DNA, 20 bp, artificial
  primer
ctgacctgaaagccgagaag

SEQ ID NO: 12 - DNA, 20 bp, artificial
  primer
ggagccatggattgcacatt

SEQ ID NO: 13 - DNA, 18 bp, artificial
  primer
ctgtctgaagaggttagg

SEQ ID NO: 14 - DNA, 21 bp, artificial
  primer
ctttgcaggtgccacttcatc

SEQ ID NO: 15 - DNA, 20 bp, artificial
  primer
accttctacaatgagctgcg

SEQ ID NO: 16 - DNA, 21 bp, artificial
  primer
agatcggagcgtctgaagatc

SEQ ID NO: 17 - DNA, 16 bp, artificial
  primer
gccaggcgcttctcaa

SEQ ID NO: 18 - DNA, 25 bp, artificial
  primer
tcttctggacccatcggccccagga

SEQ ID NO: 19 - DNA, 20 bp, artificial
  primer
ttgtggaagtgcaggttagg

SEQ ID NO: 20 - DNA, 20 bp, artificial
  primer
catgggcttcagactggtac

SEQ ID NO: 21 - DNA, 22 bp, artificial
  primer
gtgaccttcgattatgcgatca

SEQ ID NO: 22 - DNA, 21 bp, artificial
  primer
tctttcaggtcgtgttcacag

SEQ ID NO: 23 - DNA, 20 bp, artificial
  primer
agaaggtgctaacgaacagg

SEQ ID NO: 24 - DNA, 18 bp, artificial
  primer
ggcccgggaagtcactgt